(19) United States
(12) Patent Application Publication
(10) Pub. No.: US 2024/0050580 A1
(43) Pub. Date: Feb. 15, 2024

(54) COMPOSITIONS TARGETING TUMOR ASSOCIATED MACROPHAGES AND METHODS USING SAME

(71) Applicants: Rutgers, The State University of New Jersey, New Brunswick, NJ (US); Board of Regents, The University of Texas System, Austin, TX (US)

(72) Inventors: Renata Pasqualini, New Brunswick, NJ (US); Wadih Arap, New Brunswick, NJ (US); Fernanda I. Staquicini, New Brunswick, NJ (US); Amin Hajitou, Perivale London (GB); Wouter Driessen, Basel (CH); Daniela Staquicini, New Brunswick, NJ (US); Bettina Proneth, Munich (DE)

(73) Assignees: Rutgers, The State University of New Jersey, New Brunswick, NJ (US); Board of Regents, The University of Texas System, Austin, TX (US)

(21) Appl. No.: 18/268,125

(22) PCT Filed: Dec. 20, 2021
(86) PCT No.: PCT/US2021/064316

§ 371 Date: Jun. 16, 2023

Related U.S. Application Data
(60) Provisional application No. 63/128,239, filed on Dec. 21, 2020.

(51) Int. Cl. A61K 47/64; C07K 7/06; A61K 49/00; A61K 47/69; A61K 48/00; A61K 38/19; A61K 45/06; A61P 35/00; A61K 51/08

(52) U.S. Cl. CPC A61K 47/64; C07K 7/06; A61K 49/0002; A61K 47/6901; A61K 48/0041; A61K 38/191; A61K 45/06; A61P 35/00; A61K 51/08

(57) ABSTRACT

In one aspect, the present disclosure relates to tumor associated macrophage (TAM) targeting peptide. In another aspect, the present disclosure relates to a method of targeting a TAM in a subject, the method comprising administering to the subject a TAM targeting peptide attached to and/or displayed on the surface of a solid particle. In yet another aspect, the present disclosure relates to a method of treating a tumor infiltrated with a TAM in a subject, the method comprising administering to the subject a TAM targeting peptide attached to and/or displayed on the surface of a solid particle.

### CROSS-REFERENCE TO RELATED APPLICATION

The present application is entitled to priority under 35 U.S.C. § 119(e) to U.S. Provisional Patent Application No. 62/128,239, filed Dec. 21, 2020, which is hereby incorporated by reference in its entirety herein.

### SEQUENCE LISTING

The contents of the text file named “370602-7048WO1_Sequence_Listing.txt”, which was created on Sunday, Oct. 10, 2021 and is 1.25 KB in size, is hereby incorporated by reference in its entirety.

### BACKGROUND

Breast cancer is the second most common cancer type worldwide, and triple-negative breast cancer (TNBC) comprises up to ˜10-20% of all cases. TNBC is the most aggressive form of breast cancer, and it is responsible for a disproportionally large share of morbidity and mortality. These heterogeneous tumors are clinically aggressive, usually display larger sizes at initial presentation, have high pathological grade, and are likely to have lymph node involvement and early recurrence in visceral sites. The global burden of breast cancer is increasing due to a growing aging population, changes in life style, and excessive consumption of tobacco and alcohol.

TNBC is treated with multimodality therapy including neoadjuvant chemotherapy, surgery, and adjuvant radiotherapy, with selected patients receiving additional adjuvant systemic therapy. Combination chemotherapy has long been the standard therapeutic option, but checkpoint inhibitors and poly ADP-ribose polymerase (PARP) inhibitors have recently been approved in certain settings. Despite optimal management, many patients have distant metastases and poor disease outcomes. TNBC tumors are highly heterogenous and largely infiltrated by tumor associated macrophages (TAMs), a prominent component of the breast cancer microenvironment known to influence tumor progression and disease outcome.

Thus, there is a need in the art for novel compositions comprising an agent that targets TAMs and methods of treating tumors infiltrated with TAMs. The present disclosure addresses and satisfies these needs.

### SUMMARY OF THE INVENTION

As described herein, the present invention relates to tumor associated macrophage (TAM) targeting peptides. Also included are methods of targeting a TAM in a subject, the method comprising administering to the subject a TAM targeting peptide attached to and/or displayed on the surface of a solid particle. In another aspect, the present invention relates to a method of treating a tumor infiltrated with a TAM targeting peptide in a subject, the method comprising administering to the subject a TAM targeting peptide attached to and/or displayed on the surface of a solid particle.

As such, in one aspect, the invention includes a tumor associated macrophage (TAM) targeting peptide comprising at least one amino acid sequence selected from the group consisting of CSSTRESAC (SEQ ID NO:1), CRYSAARSC (SEQ ID NO:2), CRGFVVGRC (SEQ ID NO:3), and CQRALMIAC (SEQ ID NO:4).

In certain embodiments, the TAM targeting peptide comprises the amino acid sequence of SEQ ID NO:1.

In certain embodiments, the TAM targeting peptide consists of an amino acid sequence selected from the group consisting of SEQ ID NOs:1-4.

In another aspect, the invention includes a solid particle, wherein the surface of the solid particle displays the TAM targeting peptide of any one of claims 1-3, wherein the solid particle is selected from the group consisting of a bacteriophage, engineered cell, tissue fragment, nanoparticle, vesicle, dendrimer, virus-like particle, adenovirus, adeno-associated virus (AAV), adeno-associated virus phage (AAVP), and any combinations thereof.

In certain embodiments, the TAM targeting peptide is attached to and/or displayed on the surface of the solid particle.

In certain embodiments, the solid particle further comprises an agent selected from the group consisting of a therapeutic agent, biologically active molecule, imaging agent, radioactive agent, salt, peptide, protein, lipid, nucleic acid, gas, and any combinations thereof, wherein the agent is attached to and/or contained within the solid particle.

In certain embodiments, the solid particle is an AAVP.

In certain embodiments, the AAVP comprises a therapeutic or suicide gene.

In certain embodiments, the therapeutic gene is tumor necrosis factor (TNF).

In certain embodiments, the suicide gene is Herpes simplex virus thymidine kinase (HSVtk).

In another aspect, the invention includes a method of targeting a solid particle to a tumor associated macrophage (TAM) in a subject, the method comprising
administering to the subject the solid particle,
wherein the TAM targeting peptide of any one of claims 1-3 is attached to and/or displayed on the surface of the solid particle,
wherein the solid particle is selected from the group consisting of a bacteriophage, engineered cell, tissue fragment, nanoparticle, vesicle, dendrimer, virus-like particle, adenovirus, adeno-associated virus (AAV), adeno-associated virus phage (AAVP), and any combinations thereof.

In certain embodiments, the TAM targeting peptide comprises the amino acid sequence of SEQ ID NO:1.

In certain embodiments, the TAM targeting peptide consists of an amino acid sequence selected from the group consisting of SEQ ID NOs:1-4.

In certain embodiments, the solid particle further comprises an agent selected from the group consisting of a therapeutic agent, biologically active molecule, imaging agent, radioactive agent, salt, peptide, protein, lipid, nucleic acid, gas, and any combinations thereof, wherein the agent is attached to and/or contained within the solid particle.

In certain embodiments, the solid particle is an AAVP.

In certain embodiments, the AAVP comprises a therapeutic or suicide gene.

In certain embodiments, the therapeutic gene is tumor necrosis factor (TNF).

In certain embodiments, the suicide gene is Herpes simplex virus thymidine kinase (HSVtk).

In another aspect, the invention includes a method of treating, killing, and/or preventing growth of a tumor infiltrated with a tumor associated macrophage (TAM) in a subject,
the method comprising administering to the subject a solid particle, wherein the TAM targeting peptide is attached to and/or displayed on the surface of the solid particle,
wherein the solid particle is selected from the group consisting of a bacteriophage, engineered cell, tissue fragment, nanoparticle, vesicle, dendrimer, virus-like particle, adenovirus, adeno-associated virus (AAV), adeno-associated virus phage (AAVP), and any combinations thereof.

In certain embodiments, the TAM targeting peptide comprises the amino acid sequence of SEQ ID NO:1.

In certain embodiments, the TAM targeting peptide consists of an amino acid sequence selected from the group consisting of SEQ ID NOs:1-4.

In certain embodiments, the solid particle further comprises an agent selected from the group consisting of a therapeutic agent, biologically active molecule, imaging agent, radioactive agent, salt, peptide, protein, lipid, nucleic acid, gas, and any combinations thereof, wherein the agent is attached to and/or contained within the solid particle.

In certain embodiments, the solid particle is an AAVP which comprises a therapeutic or suicide gene.

In certain embodiments, the therapeutic gene is tumor necrosis factor (TNF).

In certain embodiments, the suicide gene is Herpes simplex virus thymidine kinase (HSVtk).

In certain embodiments, the method further comprises
monitoring the tumor for elevated thymidine kinase expression; and
administering a prodrug selected from ganciclovir, ganciclovir elaidic acid ester, penciclovir, acyclovir, valacyclovir, (E)-5-(2-bromovinyl)-2′-deoxyuridine, zidovuline, 2′-exo-methanocarbathymidine, and combinations thereof to the subject when elevated thymidine kinase expression is detected in the tumor.

In certain embodiments, the method further comprises evaluating the efficacy of the prodrug in treating, killing, and/or preventing growth of the tumor.

In certain embodiments, the method further comprises monitoring the tumor for elevated TNF expression.

In another aspect, the invention includes a method of treating, killing, and/or preventing growth of a tumor infiltrated with a tumor associated macrophage (TAM) in a subject, the method comprising administering to the subject an effective amount of a TAM targeting peptide.

In certain embodiments, the TAM targeting peptide comprises the amino acid sequence of SEQ ID NO:1.

In certain embodiments, the TAM targeting peptide consists of an amino acid sequence selected from the group consisting of SEQ ID NOs:1-4.

### BRIEF DESCRIPTION OF THE DRAWINGS

The following detailed description of specific embodiments of the disclosure will be better understood when read in conjunction with the appended drawings. For the purpose of illustrating the disclosure, exemplary embodiments are shown in the drawings. It should be understood, however, that the disclosure is not limited to the precise arrangements and instrumentalities of the embodiments shown in the drawings.

FIG. 1A: Immunoblotting analysis of estrogen receptor (ER), progesterone receptor (PR) and HER-2/Neu in several breast cancer cell lines confirming that EF43.fgf4 cancer cells are a triple-negative mammary cancer model. FIG. 1B: Quantitative analysis of peptide sequences obtained from the third round of in vivo phage display library screening in EF43.fgf4-tumor bearing mice.

FIGS. 2A-2K depict combinatorial targeting of the tumor cellular microenvironment in a mouse model of TNBC. FIG. 2A: A random phage display peptide library displaying CX₇C inserts (C, cysteine; X any residues) was used in vivo to select peptides homing to the microenvironment of EF43.fgf4-derived mammary tumors. Three sequential rounds of selection resulted in a pool of targeted phage particles with a 300-fold enrichment in the tumor, compared to a control organ (muscle). FIG. 2B: Binding of individual phage clones to EF43.fgf4 cells was quantified by the counting of transducing units (TU) after host bacterial infection. FIG. 2C: Binding of CSSTRESAC-phage to EF43.fgf4 tumor cells and non-malignant stromal cell subpopulations isolated from mCherry-expressing EF43.fgf4-derived mammary tumors. FIG. 2D: Relative binding of the CSSTRESAC-phage or insertless control phage to fractions eluted from a CSSTRESAC-conjugated affinity purification column. BSA was used as negative control protein. FIG. 2E: Immunoblottings developed with either anti-PDIA3 (top panel) or anti-DBP (lower panel) antibodies show the presence of both affinity-purified proteins in the experimental fraction F #5 but not in the negative control fraction F #9. Human recombinant PDIA3-GST and DBP-GST were used as control for antibody specificity. FIG. 2F: Phage binding assay confirms preferential binding of targeted CSSTRESAC-phage to the recombinant human DBP. GST and BSA were used as negative controls. FIG. 2G: Predicted structure of CSSTRESAC peptide, including a 2.0 Å-disulfide bridge between Cys1 and Cys9, as visualized with UCSF Chimera. FIG. 2H: Predicted binding conformation and orientation of CSSTRESAC relative to the crystal structure of DBP in a hydrophobicity surface view (PDB ID: 1KW2_A). Orange and blue represent hydrophobic and hydrophilic patches, respectively. FIG. 2I: Key predicted non-hydrophobic interactions between CSSTRESAC and DBP (PDB ID: 1KW2_A), including a 2.9 Å-salt bridge between Cys1 and Glu24, a 2.9 Å-salt bridge between Glu6 and Lys51, and a 2.9 Å-hydrogen bond between Ala8 and Glu24. CSSTRESAC also blocks access to Tyr48 and Ser92 (Tyr32 and Ser76 in PDB ID: 1J78), which correspond to predicted key residues of DBP interaction with 1,25-(OH)₂D₃. FIG. 2J: Crystal structure of 25-(OH)D₃ bound to DBP in a hydrophobicity surface view (PDB ID: 1J78). Orange and blue represent hydrophobic and hydrophilic patches, respectively. FIG. 2K: Binding of CSSTRESAC-phage to DBP is inhibited by the active form of vitamin D [1,25-(OH)₂D₃], but not by its corresponding vitamin D3 precursor (* represents Student's t-test, P<0.05).

FIGS. 3A-3C depict flow cytometry analysis of total cells isolated from mCherry-expressing EF43.fgf4 mammary tumors. FIG. 3A: A major component of infiltrating non-malignant cells expresses the macrophage markers CD11b and F4/80. FIG. 3B: B-lymphocytes expressing the common leukocyte antigen CD45.2, and the B-cell lineage marker CD45R were also found, although in small quantities. FIG. 3C: T-lymphocytes, as identified by the T-cell markers CD8 and CD4 were also tested but not detected.

FIG. 4: The MMTV-PyMT mouse model of breast cancer was used to confirm targeting of the CSSTRESAC-phage in vivo. Skeletal muscle was used as a negative control tissue.

FIG. 5 depicts a non-limiting MS/MS analysis.

FIGS. 6A-6G depict that PDIA3 is present on the surface of tumor associated macrophages (TAM). FIG. 6A: FACS analysis of total TAM isolated from EF43.fgf4-derived mammary tumors shows high levels of PDIA3 expression in a subpopulation of F4/80⁺CD11b⁺IL10^{high}IL12^{low} TAM. FIG. 6B: EF43.fgf4 cells do not express detectable levels of PDIA3 on their surface. FIGS. 6C-6D: PDIA3 expression in TAM and co-localization with the pan-macrophage marker CD68 as detected by immunofluorescence of tumor tissue sections from tumor-bearing mice administered iv with anti-PDIA3 antibody (FIG. 6C) or CSSTRESAC-phage (FIG. 6D). FIGS. 6E-6G: Purified TAM from EF43.fgf4 mammary tumors were established in culture and treated with either the soluble CSSTRESAC peptide, 1,25-(OH)₂D₃, or both. Controls included untreated cells, and cells treated with vehicle. Expression of anti-inflammatory (FIGS. 6E and 6G) or pro-inflammatory (FIGS. 6F and 6G) cytokines in CD11b⁺F4/80⁺ TAM was assessed by quantitative real-time PCR. Graphics represent expression fold-change relative to control cells.

FIG. 7A: An anti-PDIA3 antibody was administered iv into EF43.fgf4 tumor-bearing mice and was allowed to circulate for 5 minutes in deeply anesthetized mice before whole body perfusion through the heart. The anti-PDIA3 antibody preferentially targeted the tumor, indicating that PDIA3 is systemically accessible. FIG. 7B: The F4/80 pan-macrophage marker was used as a positive control for TAM identification. FIG. 7C: TAM isolated from EF43.fgf4 mammary tumors were established in culture and treated with the soluble CSSTRESAC peptide, 1,25-(OH)₂D₃, or both. Expression of cytokine genes was evaluated by quantitative real-time PCR. Graphics represent expression fold change relative to untreated cells.

FIGS. 8A-8E depict that targeted therapy delays growth of EF43.fgf4-derived mammary tumors. FIG. 8A: Therapeutic effect of systemic treatment of EF43.fgf4 tumor-bearing mice with soluble CSSTRESAC peptide (n=10 each experimental cohort, details in Experimental Methods). An unrelated control peptide and vehicle served as negative controls. Tumor sizes were measured by digital caliper one week after treatment initiation, and every other day afterwards. *** represents P<0.001. FIG. 8B: Treatment of tumor-bearing mice with CSSTRESAC reduces the number of PDIA3-expressing TAM (F4/80⁺CD11b⁺IL-10^{high}IL-12^{low}PDIA3+). The TAM population is represented as percentage of total non-malignant cells, as determined by flow cytometry. FIG. 8C: Gene therapy with CSSTRESAC-AAVP-HSVtk plus GCV delays tumor growth. Mice cohorts with size-matched EF43.fgf4 mammary tumors received a single systemic iv administration of targeted CSSTRESAC-AAVP-HSVtk (5×10¹⁰ TU) or control fd-AAVP-HSVtk. Mice received daily doses of GCV (80 mg/kg/day) starting at day 7 post AAVP-HSVtk administration until the end of the experiment. * represents P<0.05. FIG. 8D: Flow cytometry confirms reduction of F4/80⁺CD11b⁺IL-10^{high}IL-12^{low}PDIA3⁺ TAM in tumors from CSSTRESAC-AAVP-HSVtk-treated mice. FIG. 8E: Cytokine production by macrophages from tumors of mice treated with CSSTRESAC-AAVP-HSVtk or control groups. * represents P<0.05. Results are reported as expression fold-change relative to control group (set to 1).

FIG. 9A: FACS analysis confirming reduction of F4/80⁺CD11b⁺IL-10^{high}IL-12^{low}PDIA3+ TAM in the CSSTRESAC-treated cohort, compared to a negative control cohort (n=8 tumor-bearing mice per cohort). FIG. 9B: CD163 expression in tumor tissue sections showing reduced number of monocytes and macrophages in mice treated with soluble CSSTRESAC compared to a control peptide.

FIG. 10 depicts a heat-map representing a more extensive cytokine profile of F4/80⁺CD11b⁺IL-10^{high}IL-12^{low}PDIA3+ TAM isolated from tumors of treated and control groups.

FIG. 11 depicts a heat-map of PDIA3 gene expression in pre-defined myeloid cells from human TNBC. The heat map shows a strong association with the expression of genes characteristic of M2-polarized macrophage, markers of immunosuppression and angiogenesis (i.e., poor prognosis). The yellow box highlights cells with the highest expression of PDIA3.

FIGS. 12A-12C depicts targeted AAVP therapy delaying tumor growth. FIG. 12A: Therapeutic effect of CSSTRESAC-AAVP-TNF in EF43.fgf4-tumor-bearing mice (n=10). Cohorts of EF43.fgf4 tumor-bearing mice received two iv doses of 1×10¹⁰ TU of targeted CSSTRESAC-AAVP-TNF or the controls, insertless AAVP-TNF or CSSTRESAC-AAVP-Null (no transgene) (n=5). Untreated mice were also used as controls (n=5). Tumor sizes were measured by digital caliper every day after treatment initiation. FIG. 12B: Soluble TNF (pg/mL) in the sera of mice post-treatment. FIG. 12C: Mice body weight (g) remained unaltered post-treatment. n.s stands for no statistical significance.

### DETAILED DESCRIPTION

In one aspect, the present disclosure relates to certain peptides that target a tumor associated macrophage (TAM). In some embodiments, the peptide is attached to and/or displayed on the surface of a solid particle. One of skill in the art will understand that the solid particle can be any solid particle thought and/or known to be safe for administration to a subject. In some embodiments, the solid particle is selected from the group consisting of a phage, engineered cell, tissue fragment, nanoparticle, vesicle, dendrimer, virus-like particle (VLP), adenovirus, adeno-associated virus (AAV), adeno-associated virus phage (termed AAVP), and any combinations thereof. In other embodiments, the solid particle is a phage including, but not limited to, an adeno-associated virus phage (AAVP). In some embodiments, the solid particle further comprises a therapeutic agent, biologically active molecule, imaging agent, or radioactive agent which is contained in and/or attached to the solid particle. In some embodiments, a composition comprising the solid particle further comprises a therapeutic agent, biologically active molecule, imaging agent, or radioactive agent.

In another aspect, the present disclosure relates to a method of targeting a solid particle to a TAM within a subject, wherein the method comprises administering to the subject a TAM targeting peptide attached to and/or displayed on the surface of the solid particle. In some embodiments, the administration occurs intravenously.

In another aspect, the present disclosure relates to a method of treating, killing, and/or preventing growth of a tumor infiltrated with a TAM in a subject, the method comprising administering to the subject an effective amount of a TAM targeting peptide. In some embodiments, the administration occurs intravenously.

In yet another aspect, the present disclosure relates to a method of treating, killing, and/or preventing growth of a tumor infiltrated with a TAM in a subject, the method comprising administering to the subject a TAM targeting peptide attached to and/or displayed on the surface of a solid particle. In some embodiments, the administration occurs intravenously. In some embodiments, the TAM targeting peptide attached to and/or displayed on the surface of a solid particle treats and/or kills and/or prevents growth of the tumor by altering the local anti-tumor immune response in the subject.

In some embodiments, the solid particle is a phage, such as but not limited an AAVP, comprising any gene(s) contemplated in the art. In some embodiments, the phage comprises at least one therapeutic gene, such as but not limited to, Rb, CFTR, p16, p21, p27, p57, p73, C-CAM, APC, CTS-1, zac1, ras, DCC, NF-1, NF-2, WT-1, MEN-I, MEN-II, BRCA1, VHL, MMAC1, FCC, MCC, BRCA2, IL-1, IL-2, IL-3, IL-4, IL-5, IL-6, IL-7, IL-8, IL-9, IL-10, IL-11 IL-12, GM-CSF, G-CSF, thymidine kinase, Bax, Bak, Bik, Bim, Bid, Bad, Harakiri, Fas-L, mda-7, fus, interferon α, interferon β, interferon γ, ADP, p53, ABLI, BLC1, BLC6, CBFA1, CBL, CSFIR, ERBA, ERBB, EBRB2, ETS1, ETS2, ETV6, FGR, FOX, FYN, HCR, HRAS, JUN, KRAS, LCK, LYN, MDM2, MLL, MYB, MYC, MYCL1, MYCN, NRAS, PIM1, PML, RET, SRC, TAL1, TCL3, YES, MADH4, RB1, TP53, WT1, TNF, BDNF, CNTF, NGF, IGF, GMF, aFGF, bFGF, NT3, NT5, ApoAI, ApoAIV, ApoE, RaplA, cytosine deaminase, Fab, ScFv, BRCA2, zac1, ATM, HIC-1, DPC-4, FHIT, PTEN, ING1, NOEY1, NOEY2, OVCA1, MADR2, 53BP2, IRF-1, zac1, DBCCR-1, rks-3, COX-1, TFPI, PGS, Dp, E2F, ras, myc, neu, raf, erb, fms, trk, ret, gsp, hst, abl, ElA, p300, VEGF, FGF, thrombospondin, BAI-1, GDAIF, MCC, and combinations thereof. In some embodiments, the phage comprises at least one “suicide” gene. Exemplary suicide genes include, but are not limited to, Herpes simplex virus thymidine kinase (HSVtk), Cytosine Deaminase (CD), Purine nucleoside phosphorylase (PNP), Cytochrome p450 enzymes (CYP), Carboxypeptidases (CP), Caspase-9, Carboxylesterase (CE), Nitroreductase (NTR), Horse radish peroxidase (HRP), Guanine Ribosyltransferase (XGRTP), Glycosidase enzymes, Methionine-α,γ-lyase (MET), Thymidine phosphorylase (TP), Oxidoreductase, Cytosine deaminase, Thymidine kinase thymidilate kinase (Tdk::Tmk), deoxycytidine kinase and combinations thereof. Examples of suicide gene/prodrug combinations which may be used are Herpes simplex virus thymidine kinase (HSVtk) and ganciclovir (GCV), acyclovir, or FIAU; oxidoreductase and cycloheximide; cytosine deaminase and 5-fluorocytosine; thymidine kinase thymidilate kinase (Tdk::Tmk) and AZT; and deoxycytidine kinase and cytosine arabinoside.

In some embodiments, the solid particle is an AAVP comprising the Herpes simplex virus thymidine kinase (HSVtk) gene. In some embodiments wherein the solid particle is an AAVP comprising the HSVtk gene, the method further comprises the steps of monitoring the tumor for elevated thymidine kinase expression and administering ganciclovir (GCV) to the subject when elevated thymidine kinase expression is detected. In some embodiments, the method further comprises evaluating the efficacy of GCV in treating, killing, and/or preventing growth of the tumor.

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

### Definitions

Unless defined otherwise, all technical and scientific terms used herein have the same meaning as commonly understood by one of ordinary skill in the art to which the disclosure pertains. Although any methods and materials similar or equivalent to those described herein can be used in the practice for testing of the present disclosure, selected materials and methods are described herein. In describing and claiming the present disclosure, the following terminology will be used.

Generally, the nomenclature used herein and the laboratory procedures in cell culture, molecular genetics, pharmacology, protein chemistry, and organic chemistry are those well-known and commonly employed in the art.

Standard techniques are used for biochemical and/or biological manipulations. The techniques and procedures are generally performed according to conventional methods in the art and various general references, which are provided throughout this document.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading can occur within or outside of that particular section. All publications, patents, and patent documents referred to in this document are incorporated by reference herein in their entirety, as though individually incorporated by reference.

“About” as used herein when referring to a measurable value such as an amount, a temporal duration, and the like, is meant to encompass variations of 20% or ±10%, more preferably +5%, even more preferably +1%, and still more preferably +0.1% from the specified value, as such variations are appropriate to perform the disclosed methods.

The term “ameliorating” or “treating” means that the clinical signs and/or the symptoms associated with a disease are lessened as a result of the actions performed. The signs or symptoms to be monitored will be well known to the skilled clinician.

As used herein, by “combination therapy” is meant that a first agent is administered in conjunction with another agent. “In combination with” or “in conjunction with” refers to administration of one treatment modality (e.g. an AAVP targeted to a specific target) in addition to another treatment modality (e.g. another AAVP targeted to the same or different target). As such, “in combination with” refers to administration of one treatment modality before, during, or after delivery of the other treatment modality to the individual. Such combinations are considered to be part of a single treatment regimen or regime.

As used herein, the term “conservative sequence modifications” is intended to refer to amino acid modifications that do not significantly affect or alter the binding characteristics of the peptide containing the amino acid sequence. Such conservative modifications include amino acid substitutions, additions and deletions. Modifications can be introduced into a peptide of the disclosure by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within a peptide of the disclosure can be replaced with other amino acid residues from the same side chain family and the altered peptide can be tested for the ability to bind TAMs.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein “endogenous” refers to any material from or produced inside an organism, cell, tissue or system.

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., Sendai viruses, lentiviruses, retroviruses, adenoviruses, and adeno-associated viruses) that incorporate the recombinant polynucleotide.

Herein in certain embodiments, a “gene” refers to a nucleic acid that is transcribed. In certain aspects, the gene includes regulatory sequences involved in transcription, or message production or composition. As will be understood by those in the art, this functional term “gene” includes both genomic sequences, RNA or cDNA sequences or smaller engineered nucleic acid segments, including nucleic acid segments of a non-transcribed part of a gene, including but not limited to the non-transcribed promoter or enhancer regions of a gene. Smaller engineered gene nucleic acid segments may express, or may be adapted to express using nucleic acid manipulation technology, proteins, polypeptides, domains, peptides, fusion proteins, mutants and/or such like.

“Homologous” as used herein, refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous.

“Identity” as used herein refers to the subunit sequence identity between two polymeric molecules particularly between two amino acid molecules, such as, between two polypeptide molecules. When two amino acid sequences have the same residues at the same positions; e.g., if a position in each of two polypeptide molecules is occupied by an Arginine, then they are identical at that position. The identity or extent to which two amino acid sequences have the same residues at the same positions in an alignment is often expressed as a percentage. The identity between two amino acid sequences is a direct function of the number of matching or identical positions; e.g., if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

By the term “modified” as used herein, is meant a changed state or structure of a molecule or cell of the disclosure. Molecules can be modified in many ways, including chemically, structurally, and functionally. Cells can be modified through the introduction of nucleic acids.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

“Parenteral” administration of an immunogenic composition includes, e.g., subcutaneous (s.c.), intravenous (i.v.), intramuscular (i.m.), or intrasternal injection, or infusion techniques.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof. The peptide can be linear or branched, can comprise modified amino acids, and can be interrupted by non-amino acids. The terms also encompass an amino acid polymer modified naturally or by intervention; for example, disulfide bond formation, glycosylation, lipidation, acetylation, phosphorylation, or any other manipulation or modification, such as conjugation with a labeling component. Also included within the definition are, for example, polypeptides and proteins containing one or more analogs of an amino acid (including, for example, unnatural amino acids, and so forth), as well as other modifications known in the art. Polypeptides can occur as single chains or associated chains.

As used herein, the term “pharmaceutical composition” refers to a mixture of at least one compound useful within the disclosure with other chemical components, such as carriers, stabilizers, diluents, adjuvants, dispersing agents, suspending agents, thickening agents, and/or excipients. The pharmaceutical composition facilitates administration of the compound to an organism. Multiple techniques of administering a compound exist in the art including, but not limited to: intravenous, oral, aerosol, parenteral, ophthalmic, pulmonary, and/or topical administration.

The language “pharmaceutically acceptable carrier” includes a pharmaceutically acceptable salt, pharmaceutically acceptable material, composition or carrier, such as a liquid or solid filler, diluent, excipient, solvent or encapsulating material, involved in carrying or transporting a compound(s) of the present disclosure within or to the subject such that it can perform its intended function. Typically, such compounds are carried or transported from one organ, or portion of the body, to another organ, or portion of the body. Each salt or carrier must be “acceptable” in the sense of being compatible with the other ingredients of the formulation, and not injurious to the subject. Some examples of materials that can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; diluent; granulating agent; lubricant; binder; disintegrating agent; wetting agent; emulsifier; coloring agent; release agent; coating agent; sweetening agent; flavoring agent; perfuming agent; preservative; antioxidant; plasticizer; gelling agent; thickener; hardener; setting agent; suspending agent; surfactant; humectant; carrier; stabilizer; and other non-toxic compatible substances employed in pharmaceutical formulations, or any combination thereof. As used herein, “pharmaceutically acceptable carrier” also includes any and all coatings, antibacterial and antifungal agents, and absorption delaying agents, and the like that are compatible with the activity of the compound, and are physiologically acceptable to the subject. Supplementary active compounds can also be incorporated into the compositions.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

The term “subject” is intended to include living organisms in which an immune response can be elicited (e.g., mammals). A “subject” or “patient,” as used therein, can be a human or non-human mammal. Non-human mammals include, for example, non-human primates, and livestock and pets, such as ovine, bovine, porcine, canine, feline and murine mammals. Preferably, the subject is human.

The term “suicide gene” as used herein is defined as a nucleic acid which, upon administration of a prodrug, effects transition of a gene product to a compound which kills its host cell. Examples of suicide gene/prodrug combinations which may be used are Herpes Simplex Virus-thymidine kinase (HSVtk) and ganciclovir, acyclovir, or FIAU; oxidoreductase and cycloheximide; cytosine deaminase and 5-fluorocytosine; thymidine kinase thymidilate kinase (Tdk::Tmk) and AZT; and deoxycytidine kinase and cytosine arabinoside.

A “target site” or “target sequence” refers to a genomic nucleic acid sequence that defines a portion of a nucleic acid to which a binding molecule can specifically bind under conditions sufficient for binding to occur.

The term “therapeutic” as used herein means a treatment and/or prophylaxis. A therapeutic effect is obtained by suppression, remission, or eradication of a disease state.

To “treat” a disease as the term is used herein, means to reduce the frequency or severity of at least one sign or symptom of a disease or disorder experienced by a subject.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, Sendai viral vectors, adenoviral vectors, adeno-associated virus vectors, retroviral vectors, lentiviral vectors, and the like.

Ranges: throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Compounds and Compositions

In one aspect, the present disclosure relates to certain peptides that target a tumor-associated macrophage (TAM). In some embodiments, the present disclosure further relates to the identification of a peptide that targets a TAM. In some embodiments, the TAM targeting peptide targets the M1 population, the M2 population, and/or both populations of TAMs. In some embodiments, the TAM targeting peptide targets a TAM cell surface receptor. In some embodiments, the TAM targeting peptide targets a vitamin D receptor on the surface of a TAM cell. In some embodiments, the TAM targeting peptide binds PDIA3 on the cell surface of TAMs.

In certain embodiments, the TAM targeting peptides contemplated within the disclosure include, but are not limited to, CSSTRESAC (SEQ ID NO:1), CRYSAARSC (SEQ ID NO:2), CRGFVVGRC (SEQ ID NO:3), and/or CQRALMIAC (SEQ ID NO:4). In certain embodiments, the TAM targeting peptides of the disclosure are cyclic, wherein the cysteine at position n and the cysteine at position n+8 form a disulfide bond. In other embodiments, the transport peptides of the disclosure are not cyclic. In yet other embodiments, the TAM targeting peptide consists of CSSTRESAC (SEQ ID NO:1). In yet other embodiments, the TAM targeting peptide consists of CRYSAARSC (SEQ ID NO:2).

In yet other embodiments, the TAM targeting peptide consists of CRGFVVGRC (SEQ ID NO:3). In yet other embodiments, the TAM targeting peptide consists of CQRALMIAC (SEQ ID NO:4). In yet other embodiments, the TAM targeting peptide consists essentially of CSSTRESAC (SEQ ID NO:1). In yet other embodiments, the TAM targeting peptide consists essentially of CRYSAARSC (SEQ ID NO:2). In yet other embodiments, the TAM targeting peptide consists essentially of CRGFVVGRC (SEQ ID NO:3). In yet other embodiments, the TAM targeting peptide consists essentially of CQRALMIAC (SEQ ID NO:4). In yet other embodiments, the TAM targeting peptide comprises CSSTRESAC (SEQ ID NO: 1). In yet other embodiments, the TAM targeting peptide comprises CRYSAARSC (SEQ ID NO:2). In yet other embodiments, the TAM targeting peptide comprises CRGFVVGRC (SEQ ID NO:3). In yet other embodiments, the TAM targeting peptide comprises CSSTRESAC (SEQ ID NO:1). In yet other embodiments, the TAM targeting peptide has at least 70%, 80%, 90%, or 100% homology with the peptide of SEQ ID NO:1, SEQ ID NO:2, SEQ ID NO:3, or SEQ ID NO:4. In yet other embodiments, the TAM targeting peptide has at least 70%, 80%, 90%, or 100% identity with the peptide of SEQ ID NO:1, SEQ ID NO:2, SEQ ID NO:3, or SEQ ID NO:4.

In certain embodiments, the TAM targeting peptide contemplated in the invention is part of a polypeptide, wherein the N-terminus of the TAM targeting peptide is the N-terminus of the polypeptide (i.e., the N-terminus of the TAM targeting peptide is not coupled to other amino acids/peptides). In certain embodiments, the TAM targeting peptide contemplated in the invention is part of a polypeptide, wherein the N-terminus of the TAM targeting peptide is not the N-terminus of the polypeptide. In certain embodiments, the TAM targeting peptide contemplated in the invention is part of a polypeptide, wherein the N-terminus of the TAM targeting peptide is coupled through an amide bond to the C-terminus of a first amino acid (which is a single amino acid or the C-terminus of a (poly)peptide). In certain embodiments, the first amino acid is aspartate. In certain embodiments, the first amino acid is glutamate. In certain embodiments, the first amino acid is lysine. In certain embodiments, the first amino acid is arginine. In certain embodiments, the first amino acid is histidine. In certain embodiments, the first amino acid is alanine. In certain embodiments, the first amino acid is valine. In certain embodiments, the first amino acid is leucine. In certain embodiments, the first amino acid is isoleucine. In certain embodiments, the first amino acid is proline. In certain embodiments, the first amino acid is phenylalanine. In certain embodiments, the first amino acid is methionine. In certain embodiments, the first amino acid is tryptophan. In certain embodiments, the first amino acid is glycine. In certain embodiments, the first amino acid is asparagine. In certain embodiments, the first amino acid is glutamine. In certain embodiments, the first amino acid is cysteine. In certain embodiments, the first amino acid is serine. In certain embodiments, the first amino acid is threonine. In certain embodiments, the first amino acid is tyrosine. In certain embodiments, the first amino acid is not aspartate. In certain embodiments, the first amino acid is not glutamate. In certain embodiments, the first amino acid is not lysine. In certain embodiments, the first amino acid is not arginine. In certain embodiments, the first amino acid is not histidine. In certain embodiments, the first amino acid is not alanine. In certain embodiments, the first amino acid is not valine. In certain embodiments, the first amino acid is not leucine. In certain embodiments, the first amino acid is not isoleucine. In certain embodiments, the first amino acid is not proline. In certain embodiments, the first amino acid is not phenylalanine. In certain embodiments, the first amino acid is not methionine. In certain embodiments, the first amino acid is not tryptophan. In certain embodiments, the first amino acid is not glycine. In certain embodiments, the first amino acid is not asparagine. In certain embodiments, the first amino acid is not glutamine. In certain embodiments, the first amino acid is not cysteine. In certain embodiments, the first amino acid is not serine. In certain embodiments, the first amino acid is not threonine. In certain embodiments, the first amino acid is not tyrosine.

In certain embodiments, the TAM targeting peptide contemplated in the invention is part of a polypeptide, wherein the C-terminus of the TAM targeting peptide is the C-terminus of the polypeptide (i.e., the C-terminus of the TAM targeting peptide is not coupled to other amino acids/peptides). In certain embodiments, the TAM targeting peptide contemplated in the invention is part of a polypeptide, wherein the C-terminus of the TAM targeting peptide is not the C-terminus of the polypeptide. In certain embodiments, the TAM targeting peptide contemplated in the invention is part of a polypeptide, wherein the C-terminus of the TAM targeting peptide is coupled through an amide bond to the N-terminus of a second amino acid (which is a single amino acid or the N-terminus of a (poly)peptide). In certain embodiments, the second amino acid is aspartate. In certain embodiments, the second amino acid is glutamate. In certain embodiments, the second amino acid is lysine. In certain embodiments, the second amino acid is arginine. In certain embodiments, the second amino acid is histidine. In certain embodiments, the second amino acid is alanine. In certain embodiments, the second amino acid is valine. In certain embodiments, the second amino acid is leucine. In certain embodiments, the second amino acid is isoleucine. In certain embodiments, the second amino acid is proline. In certain embodiments, the second amino acid is phenylalanine. In certain embodiments, the second amino acid is methionine. In certain embodiments, the second amino acid is tryptophan. In certain embodiments, the second amino acid is glycine. In certain embodiments, the second amino acid is asparagine. In certain embodiments, the second amino acid is glutamine. In certain embodiments, the second amino acid is cysteine. In certain embodiments, the second amino acid is serine. In certain embodiments, the second amino acid is threonine. In certain embodiments, the second amino acid is tyrosine. In certain embodiments, the second amino acid is not aspartate. In certain embodiments, the second amino acid is not glutamate. In certain embodiments, the second amino acid is not lysine. In certain embodiments, the second amino acid is not arginine. In certain embodiments, the second amino acid is not histidine. In certain embodiments, the second amino acid is not alanine. In certain embodiments, the second amino acid is not valine. In certain embodiments, the second amino acid is not leucine. In certain embodiments, the second amino acid is not isoleucine. In certain embodiments, the second amino acid is not proline. In certain embodiments, the second amino acid is not phenylalanine. In certain embodiments, the second amino acid is not methionine. In certain embodiments, the second amino acid is not tryptophan. In certain embodiments, the second amino acid is not glycine. In certain embodiments, the second amino acid is not asparagine. In certain embodiments, the second amino acid is not glutamine. In certain embodiments, the second amino acid is not cysteine. In certain embodiments, the second amino acid is not serine. In certain embodiments, the second amino acid is not threonine. In certain embodiments, the second amino acid is not tyrosine.

Conservative amino acid replacements, i.e., replacements of one amino acid with another which has a related side chain, are also contemplated herein. Genetically-encoded amino acids are generally divided into four families: (1) acidic, i.e., aspartate, glutamate; (2) basic, i.e., lysine, arginine, histidine; (3) non polar, i.e., alanine, valine, leucine, isoleucine, proline, phenylalanine, methionine, tryptophan; and (4) uncharged polar, i.e., glycine, asparagine, glutamine, cysteine, serine, threonine, tyrosine. Phenylalanine, tryptophan, and tyrosine are sometimes classified jointly as aromatic amino acids. In general, substitution of single amino acids within these families does not have a major effect on the biological activity. The polypeptides can have one or more (e.g., 1, 2, 3, and so forth) single amino acid deletions relative to the exemplified sequences. The polypeptides can also include one or more (e.g., 1, 2, 3, and so forth) insertions relative to the exemplified sequences.

The disclosure further contemplates any nucleic acid sequences encoding any of the TAM targeting peptides of the disclosure, as well as any vectors comprising any nucleic acid sequences encoding any of the TAM targeting peptides of the disclosure, as well as any cells comprising any vector comprising any nucleic acid sequences encoding any of the TAM targeting peptides of the disclosure. The disclosure further contemplates nucleic acid sequences that have about 85%, 86%, 87%, 88%, 89%, 90%, 91%, 92%, 93%, 94%, 95%, 96%, 97%, 98%, or 99% sequence identity to the nucleic acid sequences provided herein.

In certain embodiments, at least one residue within the TAM targeting peptide, and/or at the carboxy-terminus of the TAM targeting peptide, and/or at the amino-terminus of the TAM targeting peptide is methylated, amidated, acylated (such as, but not limited to, acetylated), and/or substituted with any other chemical group without adversely affecting activity of the TAM targeting peptide within the compositions and/or methods of the disclosure. In other embodiments, the N-terminus of the TAM targeting peptide is acylated, such as but not limited to acetylated. In other embodiments, the C-terminus of the TAM targeting peptide is amidated.

In certain embodiments, the disclosure provides a solid particle, wherein the TAM targeting peptide is displayed on the surface of the solid particle. In some embodiments, the TAM targeting peptide is attached to the surface of the solid particle. In some embodiments, the TAM targeting peptide is covalently attached to the surface of the solid particle. In some embodiments, the solid particle is selected from the group consisting of a phage, engineered cell, tissue fragment, nanoparticle, vesicle, dendrimer, virus-like particle (VLP), adenovirus, adeno-associated virus (AAV), adeno-associated virus phage (termed AAVP), and any combinations thereof. In some instances, a nanoparticle has a diameter on the nanometer scale, and can vary from about 1 nm in diameter to about 5,000 nm in diameter. In some instances, a phage has a diameter that is lower than about 10 nm, such as but not limited to about 1 nm, 2 nm, 3 nm, 4 nm, 5 nm, 6 nm, 7 nm, 8 nm, 9 nm, or 10 nm. In some instances, a phage has a length that is lower than 1,000 nm, such as but not limited to about 100 nm, 200 nm, 300 nm, 400 nm, 500 nm, 600 nm, 700 nm, 800 nm, 900 nm, or 1,000 nm. In other instances, a phage has a length that is lower than 5,000 nm, such as but not limited to about 1,000 nm, 1,500 nm, 2,000 nm, 2,500 nm, 3,000 nm, 3,500 nm, 4,000 nm, 4,500 nm, or 5,000 nm. In certain embodiments, the TAM targeting peptide is attached to and/or displayed on the whole surface of the solid particle. In other embodiments, the TAM targeting peptide is attached to and/or displayed on at least a fraction of the surface of the solid particle. The solid particles can be prepared using methods known to those skilled in the art or purchased from commercial sources.

In another embodiment, the solid particle is a phage, such as but not limited to an AAVP. In some embodiments, the phage carries any gene(s) contemplated in the art. In one embodiment, the phage comprises a therapeutic gene. The present disclosure contemplates the use of a variety of different therapeutic genes. For example, genes encoding enzymes, hormones, cytokines, oncogenes, receptors, ion channels, tumor suppressors, transcription factors, drug selectable markers, toxins, and various antigens are contemplated as suitable genes for use according to the present invention. In addition, antisense and inhibitory RNA constructs derived from oncogenes are other “genes” of interest according to the present invention. A therapeutic gene or polypeptide is a gene or polypeptide which can be administered to a subject for the purpose of treating or preventing a disease. For example, a therapeutic gene can be a gene administered to a subject for treatment or prevention of cancer. Exemplary therapeutic genes include, but are not limited to, Rb, CFTR, p16, p21, p27, p57, p73, C-CAM, APC, CTS-1, zac1, ras, DCC, NF-1, NF-2, WT-1, MEN-I, MEN-II, BRCA1, VHL, MMAC1, FCC, MCC, BRCA2, IL-1, IL-2, IL-3, IL-4, IL-5, IL-6, IL-7, IL-8, IL-9, IL-10, IL-11 IL-12, GM-CSF, G-CSF, thymidine kinase, Bax, Bak, Bik, Bim, Bid, Bad, Harakiri, Fas-L, mda-7, fus, interferon α, interferon 3, interferon 7, ADP, p53, ABLI, BLC1, BLC6, CBFA1, CBL, CSFIR, ERBA, ERBB, EBRB2, ETS1, ETS2, ETV6, FGR, FOX, FYN, HCR, HRAS, JUN, KRAS, LCK, LYN, MDM2, MLL, MYB, MYC, MYCL1, MYCN, NRAS, PIM1, PML, RET, SRC, TAL1, TCL3, YES, MADH4, RB1, TP53, WT1, TNF, BDNF, CNTF, NGF, IGF, GMF, aFGF, bFGF, NT3, NT5, ApoAI, ApoAIV, ApoE, RaplA, cytosine deaminase, Fab, ScFv, BRCA2, zac1, ATM, HIC-1, DPC-4, FHIT, PTEN, ING1, NOEY1, NOEY2, OVCA1, MADR2, 53BP2, IRF-1, zac1, DBCCR-1, rks-3, COX-1, TFPI, PGS, Dp, E2F, ras, myc, neu, raf, erb, fms, trk, ret, gsp, hst, abl, ElA, p300, VEGF, FGF, thrombospondin, BAI-1, GDAIF, MCC, and combinations thereof.

In one embodiment, the phage comprises a “suicide” gene. Examples of suicide gene/prodrug combinations which may be used include, but are not limited to, Herpes simplex virus thymidine kinase (HSVtk) and ganciclovir, acyclovir, or FIAU; oxidoreductase and cycloheximide; cytosine deaminase and 5-fluorocytosine; thymidine kinase thymidilate kinase (Tdk::Tmk) and AZT; and deoxycytidine kinase and cytosine arabinoside. In certain embodiments, a suicide gene may act in the manner of a therapeutic gene by providing a therapeutic effect on a disease or medical condition as a result of the killing of its host cell. In some embodiments, the phage carries the Herpes simplex virus thymidine kinase (HSVtk) gene which enables targeted suicide therapy of tumors when used in combination with ganciclovir (GCV), ganciclovir elaidic acid ester, penciclovir (PCV), acyclovir (ACV), valacyclovir (VCV), (E)-5-(2-bromovinyl)-2′-deoxyuridine (BVDU), zidovuline (AZT), 2′-exo-methanocarbathymidine (MCT), and combinations thereof. In some embodiments, the suicide gene/prodrug combination is a combination found in Table 1.

TABLE 1
Exemplary suicide genes and prodrugs

| Suicide Gene | Prodrug |
|---|---|
| HSV thymidine kinase (TK) | Ganciclovir (GCV) |
| | Ganciclovir elaidic acid ester |
| | Penciclovir (PCV) |
| | Acyclovir (ACV) |
| | Valacyclovir (VCV) |
| | (E)-5-(2-bromovinyl)-2′- |
| | deoxyuridine (BVDU) |
| | Zidovuline (AZT) |
| | 2′-exo-methanocarbathymidine |
| | (MCT) |
| Cytosine Deaminase (CD) | 5-fluorocytosine (5-FC) |
| Purine nucleoside phosphorylase | 6-metnylpurine deoxyriboside (MEP) |
| (PNP) | fludarabine (FAMP) |
| Cytochrome p450 enzymes (CYP) | Cyclophosphamide (CPA) |
| | Ifosfamide (IFO) |
| | 4-ipomeanol (4-IM) |
| Carboxypeptidases (CP) | 4-[(2-chloroethyl)(2- |
| | mesyloxyethyl)amino]benzoyl-L- |
| | glutamic acid (CMDA) |
| | Hydroxy- and amino-aniline mustards |
| | Anthracycline glutamates |
| | Methotrexate α-peptides (MTX-Phe) |
| Caspase-9 | AP1903 (Di Stasi et al., 2011) |
| Carboxylesterase (CE) | Irinotecan (IRT) |
| | Anthracycline acetals |
| Nitroreductase (NTR) | dinitroaziridinylbenzamide CB1954 |
| | dinitrobenzamide mustard SN23862 |
| | 4-Nitrobenzyl carbamates |
| | Quinones |
| Horse radish peroxidase (HRP) | Indole-3-acetic acid (IAA) |
| | 5-Fluoroindole-3-acetic acid (FIAA) |
| Guanine Ribosyltransferase | 6-Thioxanthine (6-TX) |
| (XGRTP) | |
| Glycosidase enzymes | HM1826 |
| | Anthracycline acetals |
| Methionine-α,γ-lyase (MET) | Selenomethionine (SeMET) |
| Thymidine phosphorylase (TP) | 5′-Deoxy-5-fluorouridine (5′-DFU) |

In some embodiments, the composition comprises an imaging agent. In some embodiments, the imaging agent is a fluorescent dye or a contrast agent. In some embodiments, the fluorescent dye is rhodamine B. In some embodiments, the contrast agent is Gd-BOA. In some embodiments, the imaging agent is not attached to and/or contained within the solid particle. In another embodiment, the imaging agent is attached to and/or contained within the solid particle.

In yet another embodiment, the composition further comprises an agent selected from the group consisting of a therapeutic agent, biologically active molecule, imaging agent, radioactive agent, salt, peptide, protein, lipid, nucleic acid, gas, and any combinations thereof. In some embodiments, the agent is not attached to and/or contained within the solid particle. In another embodiment, the agent is attached to and/or contained within the solid particle.

In some embodiments, the composition comprises at least one therapeutic agent selected from a checkpoint inhibitor, a poly ADP-ribose polymerase (PARP) inhibitor, an immunomodulator, or a combination thereof.

In some embodiments, the composition comprises a therapeutic agent which is known or believed to treat a cancer associated with TAM infiltrated tumors. In some embodiments, the composition comprises a therapeutic agent which is known or believed to treat triple negative breast cancer (TNBC). Exemplary therapeutic agents include, but are not limited to, capecitabine, anthracyclines, taxanes, gemcitabine, eribulin, atezolizumab, albumin-bound paclitaxel (Abraxane), pembrolizumab, sacituzumab govitecan-hziy (Trodelvy), pamidronate (Aredia), zoledronic acid (Zometa), trabectedin (Yondelis), imatinib (Gleevec), dasatinib (Sprycel), sunitinib (Sutent), nilotinib (Tasigna), and combinations thereof. In some embodiments, the therapeutic agent is displayed on or attached to the surface of the solid particle. In another embodiment, the therapeutic agent is contained within the solid particle.

In some embodiments, the composition comprises a pharmaceutically acceptable carrier. In some embodiments, the composition comprises a solvent. In some embodiments, the solvent is an aqueous solvent. Exemplary aqueous solvents include, but are not limited to, sterile water, tap water, deionized water, distilled water, saline, and combinations thereof.

The TAM targeting peptides of the disclosure can be synthesized using chemical and biochemical methods known to those skilled in the art of chemical synthesis or peptide synthesis. The TAM targeting peptides can be attached to the surface of a solid particle using any method known to those skilled in the art. In certain embodiments, the TAM targeting peptides can be attached to the surface of a solid particle via a covalent bond. In a non-limiting example, a free amino group in the TAM targeting peptide can be attached to free carboxylate groups on the surface of a solid particle via covalent amide bonds. In a non-limiting example, a free carboxylic acid group in the TAM targeting peptide can be attached to free amino groups on the surface of a solid particle via covalent amide bonds. In other embodiments, the TAM targeting peptide can be attached to the surface of a solid particle via a non-covalent bond.

Various methods of phage display and methods for producing diverse populations of peptides are well known in the art. For example, U.S. Pat. Nos. 5,223,409; 5,622,699; 5,866,363; and 6,068,829; and JP Patent No. 4,875,497 B2; each of which is incorporated herein by reference, describe methods for preparing a phage library. The phage display technique involves genetically manipulating bacteriophage so that small peptides can be expressed on their surface [Smith, 1985, Science 228(4705):1315-1317]. In this technique, an oligonucleotide encoding a peptide of interest is inserted into a phage coat protein gene, causing the phage to “display” the protein on its outside while containing the genetic sequence for the peptide on its inside, resulting in a connection between genotype and phenotype.

It should be noted that phage display methods can be applied not only to the TAM targeting peptides but also to any peptide and/or protein that should be displayed on the surface of the phage (such as, but not limited to, a biologically active peptide and/or antigen).

Peptides and proteins contemplated in the disclosure can be prepared in several known ways, e.g., by chemical synthesis (in whole or in part), by digesting longer polypeptides using proteases, by translation from RNA, by purification from cell culture (e.g., from recombinant expression), from the organism itself (e.g., after bacterial culture, or direct from patients), and so forth. Processes for producing proteins of the disclosure are known to those skilled in the art. For example, protein production can comprise the step of culturing a host cell of the disclosure under conditions which induce protein expression.

A non-limiting method for production of peptides less than about 40 amino acids long involves in vitro chemical synthesis (Raddrizzani, et al., 2000, Briefs in Bioinformatics 14(2):121-130; Fields, et al., 1997, Principles of Peptide Synthesis. ISBN: 0387564314). Solid-phase peptide synthesis is available, such as methods based on tPoc or Fmoc chemistry (Chan, et al., 2000, Fmoc solid phase peptide synthesis. ISBN:0849368413). Enzymatic synthesis can also be used in part or in full. As an alternative to chemical synthesis, biological synthesis can be used, e.g., the polypeptides can be produced by translation. This can be carried out in vitro or in vivo. Biological methods are in general restricted to the production of polypeptides based on L-amino acids, but manipulation of translation machinery (e.g., of aminoacyl tRNA molecules) can be used to allow the introduction of D-amino acids (or of other non-natural amino acids, such as iodotyrosine or methylphenylalanine, azidohomoalanine, and so forth) (Ibba, 1996, Biotechnology and Genetic Engineering Review 13:197-216). Where D-amino acids are included, however, it is possible to use chemical synthesis. Proteins of the disclosure can have covalent modifications at the C-terminus and/or N-terminus.

Proteins useful within the disclosure can take various forms (e.g., native, fusions, glycosylated, non-glycosylated, lipidated, non-lipidated, phosphorylated, non-phosphorylated, myristoylated, non-myristoylated, monomeric, multimeric, particulate, denatured, and so forth). Proteins of the disclosure can be provided in purified or substantially purified form, i.e., substantially free from other polypeptides (e.g., free from naturally occurring polypeptides), and are generally at least about 50% pure (by weight), and usually at least about 90% pure, i.e., less than about 50%, and more preferably less than about 10% (e.g. 5%) of a composition, is made up of other expressed proteins.

Polypeptides of the disclosure can comprise a detectable label (e.g., a radioactive or fluorescent label, or a biotin label). Proteins of the disclosure can be naturally or non-naturally glycosylated (i.e., the polypeptide has a glycosylation pattern that differs from the glycosylation pattern found in the corresponding naturally occurring polypeptide).

### Methods

### Methods of Targeting a TAM in a Subject

In another aspect, the present disclosure provides a method of targeting a solid particle to a tumor associated macrophage (TAM) in a subject. In some embodiments, the method comprises administering to the subject a TAM targeting peptide attached to and/or displayed on the surface of the solid particle. In some embodiments, the method comprises administering to the subject an effective amount of a TAM targeting peptide.

In some embodiments, the subject has been diagnosed with a solid tumor cancer wherein the solid tumor is infiltrated with a TAM. In some embodiments, the subject has been diagnosed with TNBC. In other embodiments, the subject has been diagnosed with a breast cancer that is not TNBC. In some embodiments, the TAM is found in the microenvironment of a solid tumor. In some embodiments, the TAM is found in the microenvironment of a TNBC tumor. In another embodiment, the TAM is found in the microenvironment of a breast cancer tumor which is not TNBC.

The TAM targeting peptide can be any TAM targeting peptide described elsewhere herein. In some embodiments, the TAM targeting peptide is CSSTRESAC. The solid particle can be any solid particle described elsewhere herein. In some embodiments, the solid particle is a phage. In some embodiments, the solid particle is an AAVP. In some embodiments, the solid particle is a phage or AAVP which carries a therapeutic gene, a suicide gene, or a combination thereof. Exemplary therapeutic genes and suicide genes are described elsewhere herein. In some embodiments, the AAVP carries the HSVtk gene.

In some embodiments, the solid particle comprises at least one agent selected from the group consisting of a therapeutic agent, biologically active molecule, imaging agent, or radioactive agent. Exemplary agents are described elsewhere herein. In some embodiments, the agent is displayed on and/or attached to the surface of the solid particle. In another embodiment, the agent is contained within the solid particle.

In some embodiments, the TAM targeting peptide attached to and/or displayed on the surface of a solid particle is a component of a composition. In some embodiments, the composition comprises one or more pharmaceutically acceptable carriers. Exemplary pharmaceutically acceptable carriers are described elsewhere herein. In some embodiments, the composition comprises one or more agents selected from the group consisting of a therapeutic agent, biologically active molecule, imaging agent, or radioactive agent. Exemplary agents are described elsewhere herein. In some embodiments, the composition comprises an aqueous solvent.

The TAM targeting peptide attached to and/or displayed on the surface of the solid particle can be administered to the subject using any administration method known to a person of skill in the art. Exemplary administration methods are described elsewhere herein. In some embodiments, the administration occurs intravenously.

Methods of Treating a Tumor Infiltrated with a TAM in a Subject

In yet another aspect, the present disclosure provides a method of treating, killing, and/or preventing growth of a tumor infiltrated with a TAM in a subject, the method comprising administering to the subject a TAM targeting peptide attached to and/or displayed on the surface of a solid particle.

In some embodiments, the subject has been diagnosed with a solid tumor cancer wherein the solid tumor is infiltrated with TAMs. In some embodiments, the subject has been diagnosed with TNBC. In other embodiments, the subject has been diagnosed with a breast cancer that is not TNBC. In some embodiments, the TAM is found in the microenvironment of a solid tumor. In some embodiments, the TAM is found in the microenvironment of a TNBC tumor. In another embodiment, the TAM is found in the microenvironment of a breast cancer tumor which is not TNBC.

The TAM targeting peptide can be any TAM targeting peptide described elsewhere herein. In some embodiments, the TAM targeting peptide is CSSTRESAC. The solid particle can be any solid particle described elsewhere herein. In some embodiments, the solid particle is a phage In some embodiments, the solid particle is an AAVP. In some embodiments, the AAVP encodes a suicide gene, a therapeutic gene, or a combination thereof. Exemplary suicide genes and therapeutic genes are described elsewhere herein. In some embodiments, the AAVP encodes the HSVtk gene. In some embodiments, the AAVP encodes the TNF gene.

In some embodiments, the solid particle comprises at least one agent selected from the group consisting of a therapeutic agent, biologically active molecule, imaging agent, or radioactive agent. Exemplary agents are described elsewhere herein. In some embodiments, the agent is displayed on or attached to the surface of the solid particle. In another embodiment, the agent is contained within the solid particle.

In some embodiments, the TAM targeting peptide attached to and/or displayed on the surface of a solid particle is a component of a composition. In some embodiments, the composition comprises one or more pharmaceutically acceptable carriers. Exemplary pharmaceutically acceptable carriers are described elsewhere herein. In some embodiments, the composition comprises one or more agents selected from the group consisting of a therapeutic agent, biologically active molecule, imaging agent, or radioactive agent. Exemplary agents are described elsewhere herein. In some embodiments, the composition comprises an aqueous solvent.

The TAM targeting peptide attached to and/or displayed on the surface of the solid particle can be administered to the subject using any administration method known to a person of skill in the art. Exemplary administration methods are described elsewhere herein. In some embodiments, the administration occurs intravenously.

In some embodiments, the TAM targeting peptide attached to and/or displayed on the surface of a solid particle binds to the TAM in the subject. In some embodiments, the TAM targeting peptide attached to and/or displayed on the surface of a solid particle binds to a vitamin D receptor on the cell surface of the TAM. In some embodiments, the TAM targeting peptide attached to and/or displayed on the surface of a solid particle binds to PDIA3 on the cell surface of the TAM. In some embodiments, the step of administering to the subject the TAM targeting peptide attached to and/or displayed on the surface of the solid particle treats, kills, and/or prevents growth of the tumor by promoting an enhanced immune response in the subject. In some embodiments, the enhanced immune response comprises an anti-tumor immune response mediated by changes in cytokine production. In some embodiments, the TAM targeting peptide attached to and/or displayed on the surface of the solid particle treats the tumor by promoting a local inflammatory immune response in the subject. In some embodiments, the local inflammatory immune response is mediated by IL-6, IL-1β, and/or TNF-α.

In some embodiments, the step of administering to the subject a TAM targeting peptide attached to the surface of a solid particle further comprises administering to the subject a therapeutic agent. The therapeutic agent can be any agent which is known or believed to treat a tumor infiltrated with a TAM and/or a TNBC tumor. Exemplary therapeutic agents are described elsewhere herein. The therapeutic agent can be administered to the subject before, after, with the TAM targeting peptide attached to and/or displayed on the surface of the solid particle.

In some embodiments wherein the solid particle comprises a therapeutic agent or wherein the composition comprises a therapeutic agent, the method further comprises the step of evaluating the efficacy of the therapeutic agent in treating, killing, and/or preventing growth of the tumor. In other embodiments wherein the solid particle is a phage which encodes a therapeutic gene, the method further comprises the step of evaluating the efficacy of a therapeutic protein encoded by the gene in treating, killing, and/or preventing growth of the tumor. In yet other embodiments wherein the solid particle is a phage which encodes a suicide gene, the method further comprises the step of evaluating the efficacy of a prodrug administered after the phage in treating, killing, and/or preventing growth of the tumor. The efficacy of the therapeutic agent, therapeutic protein, and/or suicide gene/prodrug combination can be evaluated using any method known to a person of skill in the art. In one embodiment, an imaging agent on the attached to the surface or contained within the solid particle is used to monitor the efficacy of the therapeutic agent in treating, killing, and/or preventing growth of the tumor. In other embodiments wherein the solid particle is a phage, a composition comprising an imaging agent is administered to the subject after a TAM targeting peptide attached to and/or displayed on the surface the phage is administered to the subject. The imaging agent can be any imaging agent known to a person of skill in the art. In some embodiments, the imaging agent is a fluorescent dye or a contrast agent.

In some embodiments, the solid particle comprising a therapeutic agent, the composition comprising a therapeutic agent, or phage encoding a therapeutic gene is administered to the subject once. In other embodiments, the solid particle comprising a therapeutic agent, the composition comprising a therapeutic agent, or phage encoding a therapeutic gene is administered to the subject more than once, such as but not limited to multiple times a day, daily, every other day, weekly, and monthly. In some embodiments, the solid particle comprising a therapeutic agent, the composition comprising a therapeutic agent, or phage encoding a therapeutic gene is administered to the subject is administered until the therapeutic agent or therapeutic protein encoded by the therapeutic gene is determined to have effectively treated, killed, or prevented the growth of the tumor.

In some embodiments wherein a TAM targeting peptide attached to and/or displayed on the surface of an AAVP carrying the HSVtk gene is administered to the subject, the method further comprises the step of monitoring the tumor for elevated thymidine kinase expression. In some embodiments, the method further comprises the step of administering a prodrug selected from ganciclovir (GCV), ganciclovir elaidic acid ester, penciclovir (PCV), acyclovir (ACV), valacyclovir (VCV), (E)-5-(2-bromovinyl)-2′-deoxyuridine (BVDU), zidovuline (AZT), 2′-exo-methanocarbathymidine (MCT), and combinations thereof to the subject when elevated thymidine kinase expression is detected. In some embodiments, the prodrug is administered to the subject when maximum thymidine kinase expression is detected. Although not wishing to be limited by theory, it is believed that HSVtk converts the prodrug into a toxic product that allows for the selective elimination of TK+ cells in vivo. Therefore, it is believed that the combination of a TAM targeting peptide attached to the surface of an AAVP carrying the HSVtk gene and the prodrug will reduce tumor size, reduce the number of TAMs, and/or shift the cytokine profile toward an inflammatory response in the tumor microenvironment, thus treating the tumor.

In some embodiments, the method further comprises the step of evaluating the efficacy of the prodrug in treating, killing, and/or preventing growth of the tumor. In one embodiment, a composition comprising an imaging agent is administered to the subject before the efficacy of the prodrug is evaluated. Exemplary imaging agents are described elsewhere herein.

In some embodiments, the prodrug is administered to the subject once when elevated and/or maximum thymidine kinase expression is detected. In other embodiments, the prodrug is administered to the subject more than once, such as but not limited to, multiple times a day, daily, every other day, weekly, and monthly. In some embodiments, the prodrug is administered until it is determined to have effectively treated, killed, or prevented the growth of the tumor. In one embodiment, the prodrug is GCV.

In some embodiments wherein a TAM targeting peptide attached to and/or displayed on the surface of an AAVP carrying the TNF gene is administered to the subject, the method further comprises the step of monitoring the tumor for elevated TNF expression.

Compositions of the present disclosure can be administered in a manner appropriate to treat the tumor. The quantity and frequency of administration will be determined by such factors as the condition of the patient, and the type and severity of the patient's tumor, although appropriate dosages and schedules can be determined by clinical trials.

Compositions of the disclosure can generally be administered directly to a patient. Direct delivery can be accomplished by parenteral injection (e.g., subcutaneously, intraperitoneally, intravenously, intramuscularly, or to the interstitial space of a tissue), or by rectal, oral, vaginal, topical, transdermal, intranasal, sublingual, ocular, aural, pulmonary or other mucosal administration. In certain embodiments, the administration is intravenous.

### Pharmaceutical Compositions

Certain embodiments of the disclosure are directed to therapeutically treating an individual in need thereof. As used herein, the term “therapeutically” includes, but is not limited to, the administration of a treatment comprising a TAM targeting peptide to a subject who displays symptoms or signs of pathology, disease, or disorder, in which treatment is administered to the subject for the purpose of diminishing or eliminating those signs or symptoms of pathology, disease, or disorder.

As used herein, the term “subject” is intended to include living organisms such as mammals. Examples of subjects include, but are not limited to, horses, cows, sheep, pigs, goats, dogs, cats, rabbits, guinea pigs, rats, mice, gerbils, non-human primates, humans and the like, non-mammals, including, e.g., non-mammalian vertebrates, such as birds (e.g., chickens or ducks) fish or frogs (e.g., Xenopus), and a non-mammalian invertebrates, as well as transgenic species thereof. Preferably, the subject is a human.

### Administration/Dosage/Formulations

The regimen of administration can affect what constitutes an effective amount. The therapeutic formulations can be administered to the subject either prior to or after the onset of a disease or disorder contemplated in the disclosure. Further, several divided dosages, as well as staggered dosages can be administered daily or sequentially, or the dose can be continuously infused, or can be a bolus injection. Further, the dosages of the therapeutic formulations can be proportionally increased or decreased as indicated by the exigencies of the therapeutic or prophylactic situation.

Administration of the compositions of the present disclosure to a patient, such as a mammal, such as a human, can be carried out using known procedures, at dosages and for periods of time effective to treat a disease or disorder contemplated in the disclosure. An effective amount of the therapeutic compound necessary to achieve a therapeutic effect can vary according to factors such as the state of the disease or disorder in the patient; the age, sex, and weight of the patient; and the ability of the therapeutic compound to treat a disease or disorder contemplated in the disclosure. Dosage regimens can be adjusted to provide the optimum therapeutic response. For example, several divided doses can be administered daily or the dose can be proportionally reduced as indicated by the exigencies of the therapeutic situation. A non-limiting example of an effective dose range for a therapeutic compound of the disclosure is from about 1 and 5,000 mg/kg of body weight/per day. One of ordinary skill in the art would be able to study the relevant factors and make the determination regarding the effective amount of the therapeutic compound without undue experimentation.

In certain embodiments, the effective dose range is measured in units known to a person of skill in the art to be suitable for the description of phage doses. In some embodiments, the effective dose range for a vaccine or therapeutic compound of the disclosure is measured by transducing units (TU)/kg/dose or genome copies(GC)/kg/dose or particles/kg/dose. In some embodiments, the dosage provided to a patient is between about 10⁶-10¹² TU/kg. In some embodiments, the dosage provided to a patient is between about 10⁶-10¹² GC/kg. In some embodiments, the effective dose range is measured by colony forming units (CFU), 50% tissue culture infectious dose (TCID₅₀), plaque reduction neutralization test (PRNT), and combinations thereof.

Actual dosage levels of the active ingredients in the pharmaceutical compositions of this disclosure can be varied so as to obtain an amount of the active ingredient that is effective to achieve the desired therapeutic response for a particular patient, composition, and mode of administration, without being toxic to the patient.

The therapeutically effective amount or dose of a compound of the present disclosure depends on the age, sex and weight of the patient, the current medical condition of the patient and the progression of a disease or disorder contemplated in the disclosure.

A medical doctor, e.g., physician or veterinarian, having ordinary skill in the art can readily determine and prescribe the effective amount of the pharmaceutical composition required. For example, the physician or veterinarian could start doses of the compounds of the disclosure employed in the pharmaceutical composition at levels lower than that required in order to achieve the desired therapeutic effect and gradually increase the dosage until the desired effect is achieved.

In certain embodiments, the compositions of the disclosure are administered to the patient in dosages that range from one to five times per day or more. In other embodiments, the compositions of the disclosure are administered to the patient in range of dosages that include, but are not limited to, once every day, every two, days, every three days to once a week, and once every two weeks. It is readily apparent to one skilled in the art that the frequency of administration of the various combination compositions of the disclosure varies from individual to individual depending on many factors including, but not limited to, age, disease or disorder to be treated, gender, overall health, and other factors. Thus, the disclosure should not be construed to be limited to any particular dosage regime and the precise dosage and composition to be administered to any patient is determined by the attending physical taking all other factors about the patient into account.

In some embodiments, the compositions of the disclosure are administered to the patient while evaluating the efficacy of the compositions at treating, killing, or preventing the growth of a tumor. In some embodiments, the compositions of the disclosure are administered to the patient until it has been determined that the tumor has been treated, killed, or stopped growing. In some embodiments wherein the composition comprises a phage encoding a suicide gene, a prodrug which effects the transition of a gene product encoded by the suicide gene to a compound which kills its host cell is administered until it has been determined that the tumor has been treated, killed, or stopped growing. In some embodiments wherein the composition comprises a phage encoding a TNF gene, a HSVtk gene, a prodrug selected from ganciclovir (GCV), ganciclovir elaidic acid ester, penciclovir (PCV), acyclovir (ACV), valacyclovir (VCV), (E)-5-(2-bromovinyl)-2′-deoxyuridine (BVDU), zidovuline (AZT), 2′-exo-methanocarbathymidine (MCT), and combinations thereof is administered until it has been determined that the tumor has been treated, killed, or stopped growing. In some embodiments, the prodrug is administered daily until it has been determined that the tumor has been treated, killed, or stopped growing. In one embodiment, the prodrug is GCV. In some embodiments, the efficacy of the compositions of the disclosure are determined by monitoring the elevated expression of the gene or genes delivered by the composition. In some embodiments, the gene is HSVtk. In some embodiments, the gene is TNF. In some embodiments, both HSVtk and TNF are monitored.

It is understood that the amount of compound dosed per day can be administered, in non-limiting examples, every day, every other day, every 2 days, every 3 days, every 4 days, every 5 days, every week, every two weeks, every three weeks, every four weeks, or every month. For example, with every other day administration, a 5 mg per day dose can be initiated on Monday with a first subsequent 5 mg per day dose administered on Wednesday, a second subsequent 5 mg per day dose administered on Friday, and so on. As a second example, with every four week administration for immunization purposes, each dose can be administered every 28 days. In certain embodiments wherein the disclosed formulations or compositions are administered for immunization purposes every 28 days, serum is collected every 14 days.

In the case wherein the patient's status does improve, upon the doctor's discretion the administration of the composition of the disclosure is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced, as a function of the disease or disorder, to a level at which the improved disease is retained. In certain embodiments, patients require intermittent treatment on a long-term basis upon any recurrence of symptoms and/or infection.

The compounds for use in the method of the disclosure can be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form can be for a single daily dose or one of multiple daily doses (e.g., about 1 to 4 or more times per day). When multiple daily doses are used, the unit dosage form can be the same or different for each dose.

Toxicity and therapeutic efficacy of such therapeutic regimens are optionally determined in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index, which is expressed as the ratio between LD₅₀ and ED₅₀. The data obtained from cell culture assays and animal studies are optionally used in formulating a range of dosage for use in human. The dosage of such compounds lies in certain embodiments within a range of circulating concentrations that include the ED₅₀ with minimal toxicity. The dosage optionally varies within this range depending upon the dosage form employed and the route of administration utilized.

In certain embodiments, the compositions of the disclosure are formulated using one or more pharmaceutically acceptable excipients or carriers. In certain embodiments, the pharmaceutical compositions of the disclosure comprise a therapeutically effective amount of a compound of the disclosure and a pharmaceutically acceptable carrier.

The carrier can be a solvent or dispersion medium containing, for example, saline, buffered saline, water, ethanol, polyol (for example, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal and the like. In many cases, it is advisable to include isotonic agents, for example, sugars, sodium chloride, or polyalcohols such as mannitol and sorbitol, in the composition.

In certain embodiments, the present disclosure is directed to a packaged pharmaceutical composition comprising a container holding a therapeutically effective amount of a compound of the disclosure, alone or in combination with a second pharmaceutical agent; and instructions for using the compound to treat, prevent, or reduce one or more symptoms of a disease or disorder contemplated in the disclosure.

Formulations can be employed in admixtures with conventional excipients, i.e., pharmaceutically acceptable organic or inorganic carrier substances suitable for any suitable mode of administration, known to the art. The pharmaceutical preparations can be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They can also be combined where desired with other active agents, e.g., analgesic agents.

Routes of administration of any of the compositions of the disclosure include, oral, nasal, pulmonary, rectal, intravaginal, parenteral, buccal, sublingual, or topical. The compounds for use in the disclosure can be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration. In certain embodiments, routes of administration of any of the compositions of the disclosure include nasal, buccal, inhalational, intratracheal, intrapulmonary, and intrabronchial.

Suitable compositions and dosage forms include, for example, dispersions, suspensions, solutions, syrups, granules, beads, powders, pellets, liquid sprays for nasal or oral administration, dry powder or aerosolized formulations for inhalation, and the like. It should be understood that the formulations and compositions that would be useful in the present disclosure are not limited to the particular formulations and compositions that are described herein.

Powdered and granular formulations of a pharmaceutical preparation of the disclosure can be prepared using known methods. Such formulations can be administered directly to a subject, used, for example, to form a material that is suitable to administration to a subject. Each of these formulations can further comprise one or more of dispersing or wetting agent, a suspending agent, and a preservative. Additional excipients, such as fillers and sweetening, flavoring, or coloring agents, can also be included in these formulations.

### Oral Administration

For oral application, particularly suitable are tablets, dragees, liquids, drops, suppositories, or capsules, caplets and gelcaps. The compositions intended for oral use can be prepared according to any method known in the art and such compositions can contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets can be uncoated or they can be coated by known techniques for elegance or to delay the release of the active ingredients. Formulations for oral use can also be presented as hard gelatin capsules wherein the active ingredient is mixed with an inert diluent.

### Parenteral Administration

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intravenous, intraperitoneal, intramuscular, intrasternal injection, and kidney dialytic infusion techniques.

### Buccal, Pulmonary, Inhalational, Intranasal Administration, and So Forth

A pharmaceutical composition of the disclosure can be prepared, packaged, or sold in a formulation suitable for pulmonary administration via the buccal cavity. Such a formulation can be a liquid or dry/powder formulation comprising one or more targeting peptides of the disclosure. In some embodiments, the formulation comprises an active ingredient described elsewhere herein. In some embodiments, the particles of the dry/powder formulation have a diameter in the range from about 0.5 to about 7 micrometers, and in certain embodiments from about 1 to about 6 micrometers. Such compositions are conveniently in the form of dry powders for administration using a device comprising a dry powder reservoir to which a stream of propellant can be directed to disperse the powder or using a self-propelling solvent/powder-dispensing container such as a device comprising the active ingredient dissolved or suspended in a low-boiling propellant in a sealed container. In certain embodiments, such powders comprise particles wherein at least 98% of the particles by weight have a diameter greater than 0.5 micrometers and at least 95% of the particles by number have a diameter less than 7 micrometers. In certain embodiments, at least 95% of the particles by weight have a diameter greater than 1 micrometer and at least 90% of the particles by number have a diameter less than 6 micrometers. Dry powder compositions can include a solid fine powder diluent such as sugar and are conveniently provided in a unit dose form. See also EP Patents No. EP 02 12 753B1 and No. 1 370 318B1.

Low boiling propellants generally include liquid propellants having a boiling point of below 65° F. at atmospheric pressure. Generally the propellant can constitute 50 to 99.9% (w/w) of the composition, and the active ingredient can constitute 0.1 to 20% (w/w) of the composition. The propellant can further comprise additional ingredients such as a liquid non-ionic or solid anionic surfactant or a solid diluent (in certain embodiments having a particle size of the same order as particles comprising the active ingredient).

Pharmaceutical compositions of the disclosure formulated for pulmonary delivery can also provide the active ingredient in the form of droplets of a solution or suspension. Such formulations can be prepared, packaged, or sold as aqueous or dilute alcoholic solutions or suspensions, optionally sterile, comprising the active ingredient, and can conveniently be administered using any nebulization or atomization device. Such formulations can further comprise one or more additional ingredients including, but not limited to, a flavoring agent such as saccharin sodium, a volatile oil, a buffering agent, a surface active agent, or a preservative such as methylhydroxybenzoate. The droplets provided by this route of administration in certain embodiments have an average diameter in the range from about 0.1 to about 200 micrometers.

The pharmaceutical composition of the disclosure can be delivered using an inhalator such as those recited in U.S. Pat. No. 8,333,192 B2, which is incorporated herein by reference in its entirety.

The formulations described herein as being useful for pulmonary delivery are also useful for intranasal delivery of a pharmaceutical composition of the disclosure.

Another formulation suitable for intranasal administration is a coarse powder comprising the active ingredient and having an average particle from about 0.2 to 500 micrometers. Such a formulation is administered in the manner in which snuff is taken, i.e., by rapid inhalation through the nasal passage from a container of the powder held close to the nares. Formulations suitable for nasal administration may, for example, comprise from about as little as 0.1% (w/w) and as much as 100% (w/w) of the active ingredient, and can further comprise one or more of the additional ingredients described herein.

### Additional Administration Forms

Additional dosage forms of this disclosure include dosage forms as described in U.S. Pat. Nos. 6,340,475; 6,488,962; 6,451,808; 5,972,389; 5,582,837; and 5,007,790. Additional dosage forms of this disclosure also include dosage forms as described in U.S. Patent Applications Nos. 20030147952; 20030104062; 20030104053; 20030044466; 20030039688; and 20020051820. Additional dosage forms of this disclosure also include dosage forms as described in PCT Applications Nos. WO 03/35041; WO 03/35040; WO 03/35029; WO 03/35177; WO 03/35039; WO 02/96404; WO 02/32416; WO 01/97783; WO 01/56544; WO 01/32217; WO 98/55107; WO 98/11879; WO 97/47285; WO 93/18755; and WO 90/11757.

### Controlled Release Formulations and Drug Delivery Systems

In certain embodiments, the formulations of the present disclosure can be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time can be as long as a month or more and should be a release which is longer that the same amount of agent administered in bolus form.

For sustained release, the compounds can be formulated with a suitable polymer or hydrophobic material that provides sustained release properties to the compounds. As such, the compounds for use the method of the disclosure can be administered in the form of microparticles, for example, by injection or in the form of wafers or discs by implantation.

In certain embodiments of the disclosure, the compounds of the disclosure are administered to a patient, alone or in combination with another pharmaceutical agent, using a sustained release formulation.

The term delayed release is used herein in its conventional sense to refer to a drug formulation that provides for an initial release of the drug after some delay following drug administration and that may, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

Those skilled in the art will recognize, or be able to ascertain using no more than routine experimentation, numerous equivalents to the specific procedures, embodiments, claims, and examples described herein. Such equivalents were considered to be within the scope of this disclosure and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present disclosure. Moreover, all values that fall within these ranges, as well as the upper or lower limits of a range of values, are also contemplated by the present application.

The following examples further illustrate aspects of the present disclosure. However, they are in no way a limitation of the teachings or disclosure of the present disclosure as set forth herein.

The practice of the present disclosure employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, 4th edition (Sambrook, 2012); “Oligonucleotide Synthesis” (Gait, 1984); “Culture of Animal Cells” (Freshney, 2010); “Methods in Enzymology” “Handbook of Experimental Immunology” (Weir, 1997); “Gene Transfer Vectors for Mammalian Cells” (Miller and Calos, 1987); “Short Protocols in Molecular Biology” (Ausubel, 2002); “Polymerase Chain Reaction: Principles, Applications and Troubleshooting”, (Babar, 2011); “Current Protocols in Immunology” (Coligan, 2002). These techniques are applicable to the production of the polynucleotides and polypeptides of the disclosure, and, as such, can be considered in making and practicing the disclosure.

It should be understood that the method and compositions that would be useful in the present disclosure are not limited to the particular formulations set forth in the examples. The following examples are put forth so as to provide those of ordinary skill in the art with a complete disclosure and description of how to make and use the cells, expansion and culture methods, and therapeutic methods of the disclosure, and are not intended to limit the scope of what the inventors regard as their disclosure.

### EXPERIMENTAL EXAMPLES

The disclosure is now described with reference to the following Examples. These Examples are provided for the purpose of illustration only, and the disclosure is not limited to these Examples, but rather encompasses all variations that are evident as a result of the teachings provided herein.

### Materials and Methods

### Antibodies and Recombinant Proteins

Anti-PDIA3, anti-IL-10, anti-IL-12, and anti-F4/80 were purchased from BD Pharmingen and were used in flow cytometry and immunofluorescence. Immunoblottings were performed with antibodies purchased from Sigma (Glutathione S-transferase, PDIA3, and DBP), Abcam (ER), Cell Signaling (PgR), and R&D Systems (HER2). Taqman assays for real-time PCR quantification of cytokines were purchased from Applied Biosystems. Recombinant proteins (DBP and PDIA3), cholecalciferol, and calcitriol were all acquired from Abcam. Fluorescence-conjugated secondary antibodies were purchased from Jackson Immunoresearch. Peptides were custom synthesized by PolyPeptide Laboratories to the desired specifications.

### Cells Lines and Tissue Culture

Mouse mammary EF43.fgf4 cells were maintained in Dulbecco's modified Eagle's medium (DMEM) supplemented with 10% fetal bovine serum (FBS), 5 ng/ml mouse epithelial growth factor (EGF), 1 pg/ml bovine insulin, and antibiotics. Cells were maintained at 37° C. and 5% C02.

### Animals and Experimental Tumor Models

Eight-week-old female nude (nu/nu) mice and immunocompetent BALB/c mice were housed in institutions' animal facilities. All animal procedures were reviewed and approved by the corresponding Institutional Animal Care and Use Committee (IACUC) at each institution.

Mouse EF43.fgf4 cells were implanted in the mammary fat pads of immunocompetent BALB/c mice. Tumor-bearing mice were sorted into experimental size-matched cohorts when established tumors reached ˜200 mm³. These procedures were conducted in accordance with the Guide for the Care and Use of Laboratory Animals published by the US National Institutes of Health and approved by the local ethics review board.

PyMT [strain FVB/N-TgN (MMTVPyVT)₆₃₄-Mul] were obtained from Jackson Laboratory (Bar Harbor, ME, USA). All procedures involving PyMT mice were conducted in accordance with the German Animal Protection Code and approval was granted by the local ethics review board (Hamburg, Germany). PyMT transgenic mice genotyping was performed through blood samples collected from the retrobulbar venous plexus under anesthesia (2% isoflurane, 98% oxygen), as described in the literature.

Treatment of tumor-bearing mice with a single-dose of CSSTRESAC-AAVP-HSVtk or control fd-AAVP-HSVtk (5×10¹⁰ TU per mouse) was followed by daily intraperitoneal (ip) administrations of GCV at 80 mg/kg/day. Tumor sizes were measured every other day with a digital caliper and plotted as tumor volume (mm³).

For AAVP-TNF studies using immunocompetent animals, six to eight-week-old female immunocompetent BALB/c mice were housed in the animal facilities of Rutgers Cancer Institute of New Jersey at Rutgers University New Jersey Medical School. EF43.fgf4 syngeneic mouse mammary gland tumor cells were implanted in the flank of BALB/c mice (1×10⁶ cells per mouse). Tumor-bearing mice were sorted into experimental size-matched cohorts when established tumors reached ˜200 mm³. Tumor-bearing mice were treated with two doses of CSSTRESAC-AAVP-TNF (1×10¹⁰ TU) or the controls, insertless AAVP-TNF (1×10¹⁰ TU) or CSSTRESAC-AAVP-Null (empty, no transgene) (1×10¹⁰ TU), administered intravenously on days 4 and 7 post-tumor implantation. Tumor sizes were measured every day with a digital caliper and plotted as tumor volume (mm³).

### Phage Display Methodology

The Biopanning and Rapid Analysis of Selective Interactive Ligands (BRASIL) methodology was used to test binding of phage to cultured cells. For phage binding to the candidate receptors PDIA3 and DBP, individual microtiter wells of 96-well plates were coated overnight (ON) with 1 μg/ml of recombinant proteins, followed by blocking with BSA and incubation with 10⁹ TU of insertless phage or CSSTRESAC-phage for 1 hour at room temperature (RT). GST and BSA were used as control proteins. Bound phage were recovered by log-phase infection of host bacteria (200 μl E. coli K91Kan). Competitive binding of CSSTRESAC-phage and 1,25-(OH)₂D₃ to DBP was performed by using the same experimental protocol. Competition was performed in wells pre-incubated with 3 nM or 30 nM of either 1,25-(OH)₂D₃ or cholecalciferol.

Combinatorial phage display selections in vivo in tumor-bearing mice were performed as described in the literature. In brief, animals received 10⁹ TU iv of an unselected phage display random peptide library (displaying the insert CX₇C). Tumors and control organs were collected after 24 hours of systemic circulation. For homing of individual phage clones in vivo, tumor-bearing mice were deeply anesthetized with 1-2% isoflurane and received 10⁹ TU of targeted phage or insertless control phage, both administered iv side-by-side. Phage particles were recovered from tissue samples by bacterial infection and processed as described in the literature.

### Peptide Affinity Chromatography

Receptor candidates were isolated by using an affinity chromatography CARBOXYLINK™ column (ThermoFisher Scientific) conjugated with the synthetic CSSTRESAC peptide. Protein extracts (10 mg/purification) were added to peptide conjugated columns and incubated ON at 4° C. under constant gentle agitation. After extensive washes, bound proteins were eluted with an excess of soluble CSSTRESAC (SEQ ID NO:1) peptide followed by elution in low pH glycine buffer. Contaminants including detergents, salts, lipids, phenolics, and nucleic acids were removed through a 2-D clean-up kit from GE Healthcare Life Sciences. Proteins were re-suspended in rehydration buffer (8 M urea, 2% CHAPS, 40 mM DTT, 0.5% IPG buffer, 0.002% bromophenol blue) and 2-D gel electrophoresis was performed by using the ZOOM® IPGRunner™ System (Life Technologies). The final gel was stained with SYPRO® Ruby Protein Gel Stain (Life Technologies) and imaged in a 300 nm ultraviolet transilluminator. Unique bands were excised from the SDS gels and digested with trypsin. LC-MS/MS analysis was performed.

To test purified fractions for the presence of candidate receptors, control and experimental fractions were immobilized on individual microtiter wells of 96-well plates ON at 4° C. Wells were blocked with phosphate-buffered saline (PBS) containing 3% BSA for 1 hour at RT and incubated with 10⁹ TU of insertless phage or CSSTRESAC-phage. After extensive washing with PBS, bound phage particles were recovered by infection of host bacteria.

### Peptide Structure Prediction and Docking

The peptide sequence of CSSTRESAC (SEQ ID NO:1) was entered into PEP-FOLD2 with a designated disulfide bridge between Cys1 and Cys9 (to ensure the cyclic peptide configuration) and 100- and 200-run simulations were applied. The best-fit model containing a disulfide bridge between Cys1 and Cys9 based on sOPEP energy (i.e., the negative value with greatest absolute value) was selected as the structure for further experimentation. By using the UCSF Chimera, a PDB file with CSSTRESAC (SEQ ID NO:1) positioned adjacent to human DBP (PDB ID: 1KW2_A)₂₇ in roughly the same location as 25-(OH)D₃ in its complex with human DBP (PDB ID: 1J78) was generated and inputted into Rosetta FlexPepDock. The top generated model according to energy scoring, a revised version of Rosetta full-atom and coarse-grained energy functions, with CSSTRESAC (SEQ ID NO:1) bound to the same binding pocket as 25-(OH)D₃ was selected for analysis. Interacting residues of CSSTRESAC (SEQ ID NO:1) and DBP were analyzed via UCSF Chimera.

### Immunohistochemistry, Immunofluorescence, and Flow Cytometry

For immunohistochemistry and immunofluorescence, the anti-PDIA3 antibody was administered iv into the tail vein of EF43.fgf4 tumor-bearing BALB/c mice. After 5 minutes, the mice were killed and perfused through the heart. Tumors and control organs were collected and either quickly-frozen in liquid nitrogen or preservative-fixed, and paraffin-embedded. The presence of the anti-PDIA3 antibody in tissue sections was verified by detection with a secondary antibody conjugated to horseradish peroxidase (HRP) or were stained for the presence of macrophages with an anti-CD68 antibody conjugated to FITC. For flow cytometry, whole EF43.fgf4 tumors were dissected out from tumor-bearing BALB/c mice and single cell suspensions were prepared by tumor mincing. The single cell suspension was washed with PBS containing 5% FBS and 0.01% NaN3. Cell suspensions were aliquoted into 12×75 mm flow cytometry tubes as 5×10⁵ cells per tube and ice-cold incubated for 15 minutes with an Fc receptor blocking agent, followed by antibodies against PDIA3, F4/80, IL-10 and IL-12. Cells were incubated on ice for 30 minutes, followed by washes and secondary antibodies.

### Quantitative Real-Time PCR

Three sets of total RNA (RNeasy Mini Kit, Qiagen) were independently isolated from cultured macrophages, or fresh macrophages isolated directly from tumors. DNA synthesis was performed with the GOSCRIPT™ Reverse Transcription System (Promega) by using oligodT for reverse transcription. Gene expression was analyzed with the use of Tagman probes (Applied Biosystems) in a 7500 Fast Real-Time PCR System instrument (Applied Biosystems) and three sets of endogenous control genes: 18S and GAPDH and GUSB1.

### Macrophage Isolation and Tissue Culture

TAM were obtained directly from EF43.fgf4 tumors. Tissue digestion was performed in collagenase A in serum-free DMEM (1 mg/mL) for 20 minutes at 37° C., followed by filtering through 70 μm nylon cell strainers and centrifugation. Macrophages were enriched by magnetic bead separation of CD11b-positive cells (Miltenyi Biotec) and either used for RNA extraction or cultured in 6-well plates containing DMEM (Gibco) supplemented with 20% FBS (Sigma) and 50 ng/ml of M-CSF (R&D Systems). A homogeneous population of adherent macrophages (namely, >99% CD11b+F480+) was obtained after 7 days in culture.

PDIA3 Gene Expression in Human Single Cells from TNBC Patients

In order to evaluate the expression levels of PDIA3 mRNA in human breast cancer samples, the clinical and scRNA-seq data were obtained from a publicly available single cell database from TNBC patients (BC07-BC11). The scRNA-seq datasets were reported as TPM and were assessed through the GEO repository (accession number GSE75688). Gene expression levels of 35 pre-defined myeloid cells were retrieved and distributed into three groups according to PDIA3 expression levels (set as high, medium, or low). A heat-map was generated to show potential associations between PDIA3 and gene pathways characteristic of macrophages.

### Statistical Analysis

Comparisons among the groups were assessed by One-way ANOVA with SigmaStat (SPSS Inc.) and GraphPad Prism (GraphPad Software Inc.). Statistical significance was set at a P-value of <0.05 unless otherwise specified. Normally distributed data are shown as bar graphs with means±standard deviation (SD) or standard error of the mean (SEM) as indicated, whereas not normally distributed data are shown in box-and-whiskers plots: the boxes define the 25^{th} and 75^{th} percentiles, a line denotes the median and error bars define the 10^{th} and 90^{th} percentiles.

### Selected Results

TNBC is the most aggressive form of breast cancer, and it is responsible for a disproportionally large share of morbidity and mortality. These tumors are highly heterogenous and largely infiltrated tumor associated macrophages (TAMs), a prominent component of the breast cancer microenvironment known to influence tumor progression and disease outcome.

Immunomodulators are among the best available investigational drugs for this tumor subtype, based on the premise that manipulation of the local and/or distant immune responses may ultimately represent a viable treatment approach. A biological hallmark of TNBC is an immunosuppressive tumor microenvironment that fosters tumor growth and metastatic spread through the suppression of tumor-infiltrating lymphocytes and secretion of immunoinhibitory cytokines, mainly by TAMs. TAMs are classically divided into two major populations, M1 and M2, representing the extremes of a broad activation state spectrum. The M1 population is associated with anti-tumor activity while the M2 population with tumor progression. Such biological behavior in breast cancer has made them potentially attractive targets for therapeutic intervention. In fact, TAM-targeting drugs are currently in clinical trials but have not yet been approved for clinical practice. Herein, in vivo and in vitro phage display technologies were combined to search for new therapeutic options to treat TNBC.

### Combinatorial Phage Display Screening In Vivo Reveals Tumor Microenvironment-Binding Peptides in a Mouse Model of TNBC

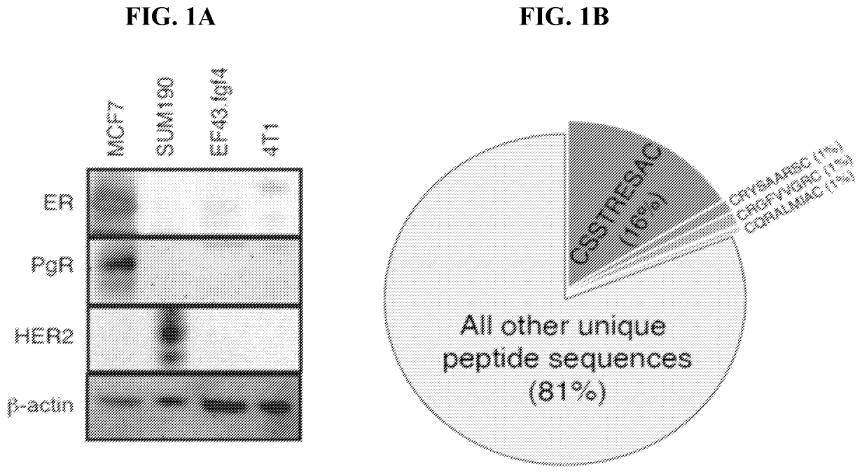

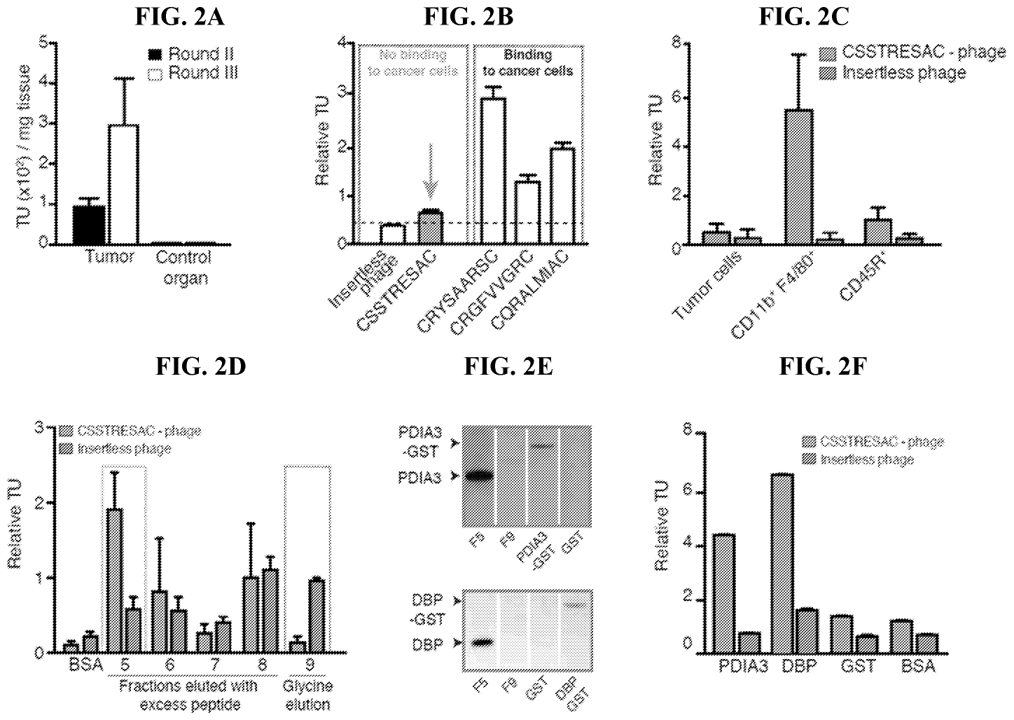

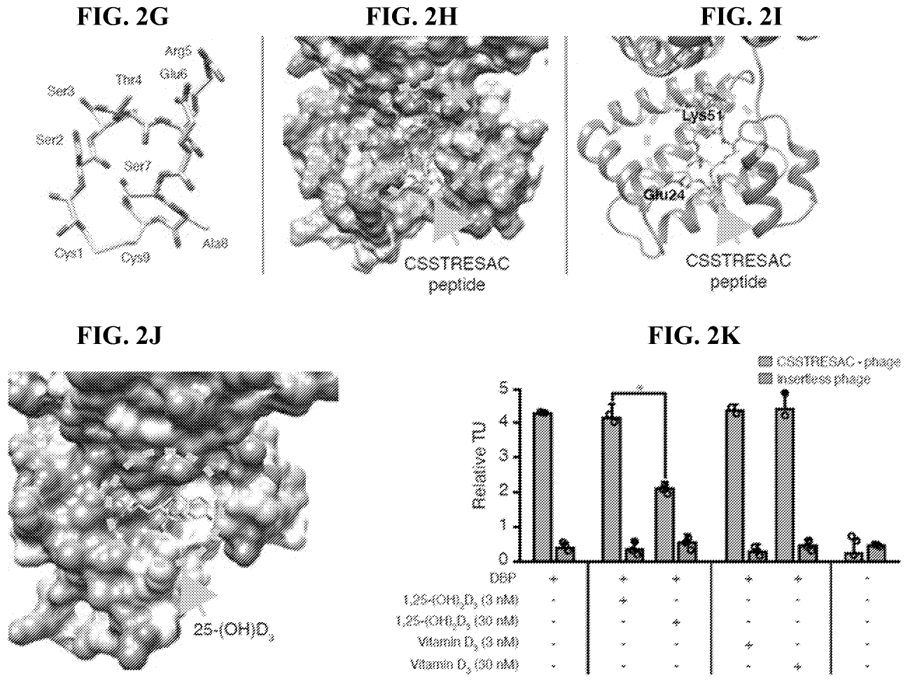

A phage display-based approach was used to identify homing peptides that target TAM in TNBC. The EF43.fgf4 syngeneic mouse mammary gland tumor is highly infiltrated by TAM and also serves as an immunocompetent TNBC model since EF43.fgf4 cells do not express the estrogen receptor, progesterone receptor, or Erbb2/Neu (FIGS. 1A-1B). A random phage peptide library was first administered intravenously (iv) in immunocompetent female BALB/c mice with established EF43.fgf4-derived mammary fat pad tumors. Phage particles were recovered from tumors after 24 hours, re-amplified, and subjected to two additional rounds of in vivo selection. After the third round, the pool of tumor-homing phage showed an ˜300-fold enrichment relative to normal tissues (FIG. 2A). Bioinformatic analysis of peptides targeting the whole tumor revealed four sequences above an experimental threshold (set at 1%): CSSTRESAC (SEQ ID NO:1), CRYSAARSC (SEQ ID NO:2), CRGFVVGRC (SEQ ID NO:3), and CQRALMIAC (SEQ ID NO:4). Notably, the dominant peptide CSSTRESAC (SEQ ID NO:1) was more strongly enriched (16-fold) than each of the other three peptides (FIG. 2B). The four selected peptides were next individually evaluated based on absence of binding to EF43.fgf4 cells in vitro (FIG. 2B). With a standard cell binding assay, it was found that the peptides CRGFVVGRC (SEQ ID NO:3), CQRALMIAC (SEQ ID NO:4), and CRYSAARSC (SEQ ID NO:2) bound to EF43.fgf4 cells, whereas the peptide CSSTRESAC (SEQ ID NO:1) did not (FIG. 2B). While not wishing to be limited by theory, this may indicate that CSSTRESAC (SEQ ID NO:1) recognizes non-malignant stromal cells within the tumor microenvironment. In a non-limiting aspect, the peptides CRGFVVGRC, (SEQ ID NO:3) CQRALMIAC (SEQ ID NO:4), and CRYSAARSC (SEQ ID NO:2) were not studied further.

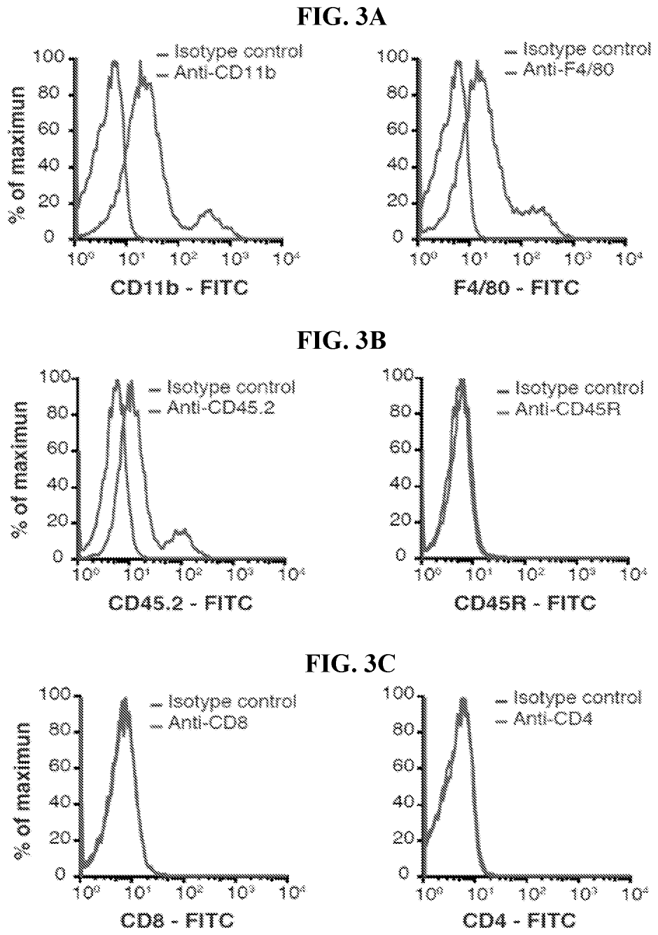

To identify the non-malignant cellular component(s) targeted by CSSTRESAC-phage, binding to subcellular populations freshly isolated from engrafted tumors was tested. mCherry-expressing EF43.fgf4 cells were FACS-sorted from whole tumors. The remaining cells were subsequently FACS-sorted based on expression of CD45 (Leukocyte Common Antigen, LCA) and F4/80, respectively. Similar to human breast cancers known to be highly infiltrated by macrophages, the macrophage population (CD11b⁺F4/80⁺) constituted a large portion of the non-malignant cellular component of EF43.fgf4-derived mammary tumors, followed by a lesser population of B lymphocytes (CD45R⁺). T-lymphocytes (CD8⁺ or CD4⁺) were not detected (FIGS. 3A-3C). Binding assays to each of these cell subpopulations showed that CSSTRESAC-phage particles bound specifically to CD11b⁺F4/80⁺ *macrophage; binding to tumor-isolated EF43.fgf4 cells and CD45R⁺ cells were at background levels (FIG. 2C). Based on these results, it was concluded that CSSTRESAC-phage particles target a TAM cell surface receptor.

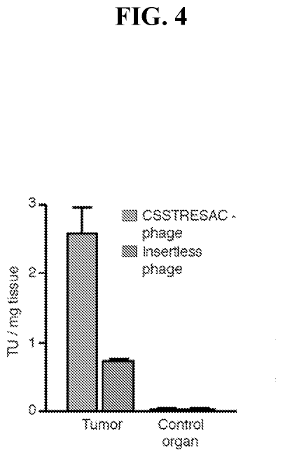

Although it was shown that the CSSTRESAC-phage targeted TAM in a syngeneic TNBC model, the possibility was considered that it might be able to target the tumor microenvironment in other experimental models of non-TNBC breast cancer also known to be infiltrated by TAM. First, CSSTRESAC-phage homing was tested in the mouse mammary tumor virus-polyoma middle T-antigen (MMTV-PyMT) transgenic model of breast cancer. Binding of the CSSTRESAC-phage to MMTV-PyMT tumors was higher compared to a control organ (˜3-fold) or to a negative control phage (˜2.5-fold) (FIG. 4).

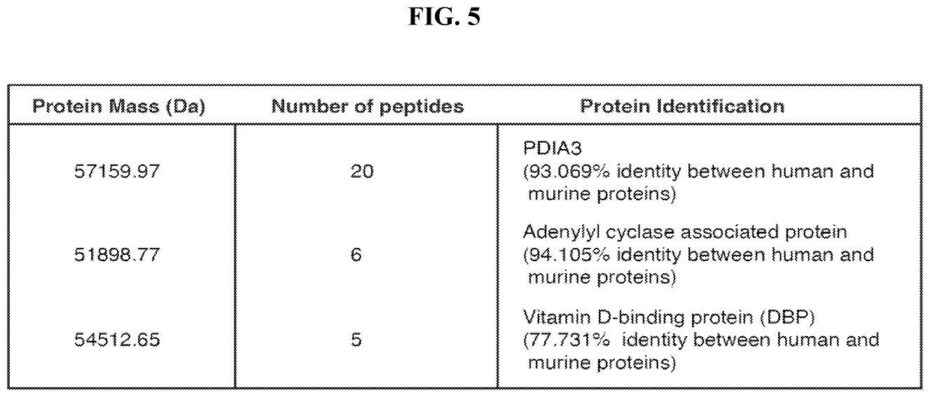

Next, peptide affinity chromatography was used to identify the cell surface receptor(s) in TAM targeted by the CSSTRESAC (SEQ ID NO:1) peptide. Interacting proteins were eluted through an excessive amount of soluble CSSTRESAC (SEQ ID NO:1) peptide and subsequently control acidic glycine buffer. Binding assays were used to identify eluted fractions containing the highest concentrations of receptor(s) (FIG. 2D). Proteins present in fraction (F) #5 (positive experimental fraction) and F #9 (negative control fraction) were resolved by sodium dodecyl sulfate-polyacrylamide gel electrophoresis (SDS-PAGE), and differential protein bands were subjected to in tandem mass spectrometry fragmentation (LS-MS/MS) for protein identification (FIG. 5). Notably, immunoblotting of eluted fractions revealed the presence of two vitamin D-binding receptor candidates: protein disulfide-isomerase A3 (PDIA3; also known as glucose-regulated protein-58 kDa, GRP58; endoplasmic reticulum protein of 57 kDa, ERp57; and membrane-associated rapid response to steroid-binding, 1,25D₃-MARRS) (FIG. 2E, top panel) and vitamin D-binding protein (DBP) (FIG. 2E, bottom panel). In vitro binding assays to recombinant PDIA3 and DBP confirmed preferential binding of CSSTRESAC-phage relative to the negative control insertless phage (FIG. 2F).

### CSSTRESAC (SEQ ID NO:1) Mimics Active Vitamin D

PDIA3 and DBP both bind to vitamin D, thereby suggesting that CSSTRESAC (SEQ ID NO:1) might be structurally similar to vitamin D. Thus, computational molecular modeling was applied to determine whether the peptide CSSTRESAC (SEQ ID NO:1) would show conformational similarities to vitamin D (FIGS. 2G-2J). The structure of CSSTRESAC (SEQ ID NO:1) was modeled with a de novo peptide structure prediction tool (PEP-FOLD2) (FIG. 2G). Next, Rosetta FlexPepDock was used to identify putative binding site(s) for CSSTRESAC (SEQ ID NO:1) on the surface of DBP. Because the 3D structure of the DBP/1,25-(OH)₂D₃ complex was not available when this work was performed, a 2.3 Å-resolution X-ray crystal structure of the unliganded form of human DBP (PDB ID: 1KW2_A) was used. To initiate the docking calculation, CSSTRESAC (SEQ ID NO:1) was pre-positioned in the vicinity of the known binding site for 25-(OH)D₃, (and likely 1,25-(OH)₂D₃ based on previous computational modeling), visualized in the 2.1 Å-resolution X-ray crystal structure of a liganded form of human DBP (PDB ID: 1J78). The molecule 25-(OH)D₃, also known as calcidiol, binds at the base of a deep, largely hydrophobic pocket on the surface of domain I of DBP (FIG. 2J). The computed model of the DBP/CSSTRESAC complex revealed a potential binding site for CSSTRESAC (SEQ ID NO:1) at the opening of the hydrophobic pocket. The computed model indicates in a non-limiting embodiment that the largely hydrophilic peptide interacts with two superficial residues adjacent to the hydrophobic pocket, including Glu24 and Lys51 (FIGS. 2H and 2I). The outcome of the Rosetta FlexPepDock calculations suggests that although CSSTRESAC (SEQ ID NO:1) binds at a similar site on the surface of DBP as 1,25-(OH)₂D₃ and its metabolite calcidiol, it is unlikely to interact more tightly and should be competitively displaced by the natural ligands of the receptor protein (FIGS. 2I and 2J). Indeed, experimental binding of CSSTRESAC-phage to immobilized DBP was reduced (Student's t-test, P<0.05) by increasing amounts of 1,25-(OH)₂D₃ but not by the non-active precursor vitamin D₃ (FIG. 2K), a biochemical finding consistent with the computational model.

### PDIA3 is a Receptor of the CSSTRESAC (SEQ ID NO:1) Peptide and a Novel Molecular Marker of TAMs

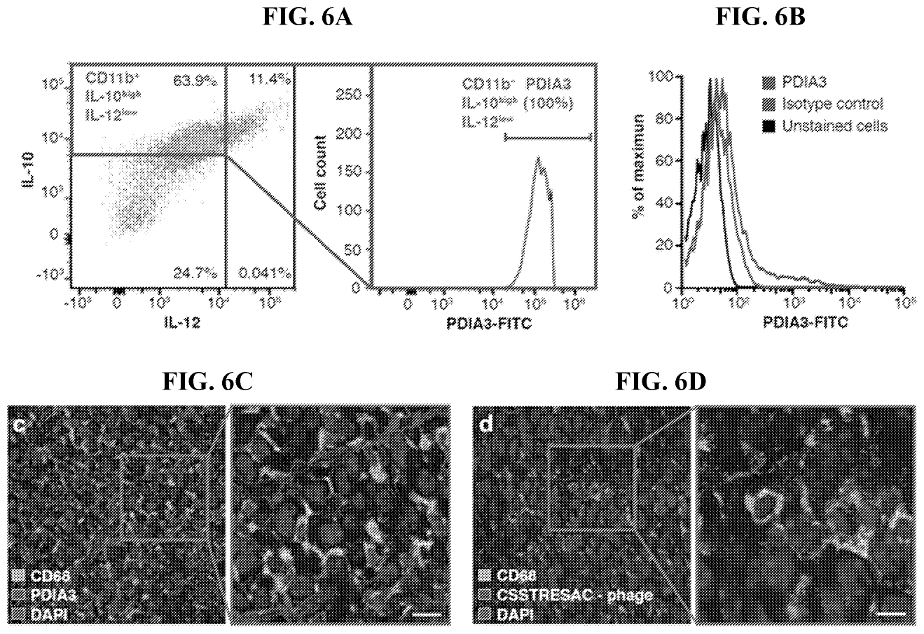

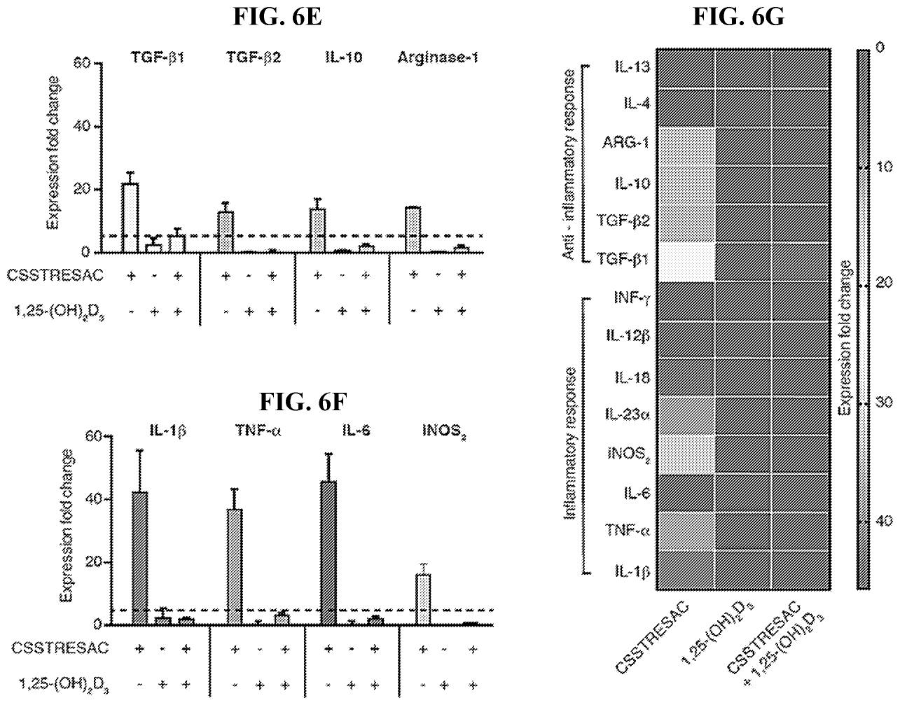

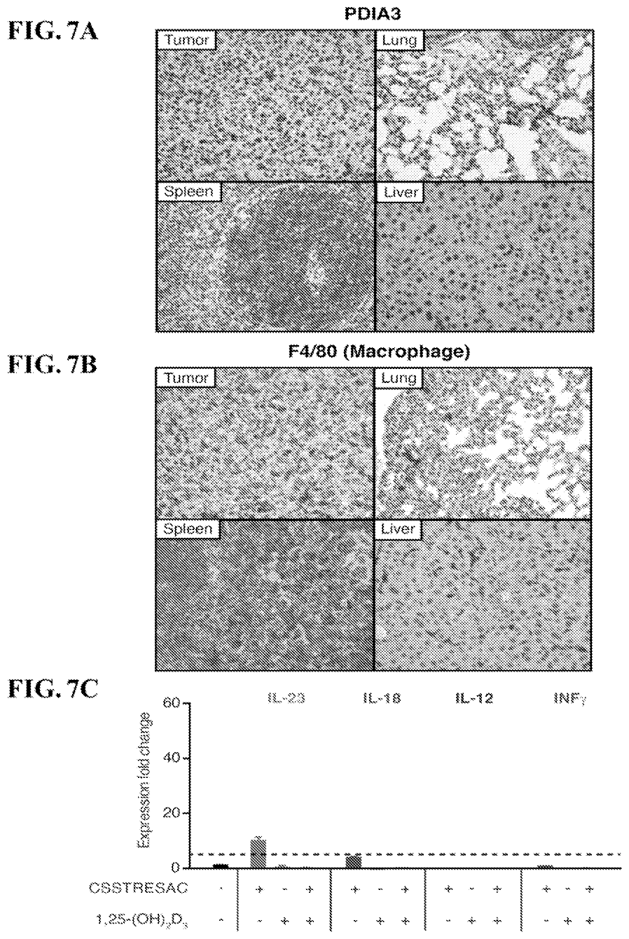

Despite the fact that binding of CSSTRESAC (SEQ ID NO:1) to DBP is strongly suggested by the structural modeling, DBP is a circulating serum protein and thus unlikely to function as an integral cell surface receptor. Therefore, it was reasoned that the membrane-bound receptor candidate PDIA3 would likely be the cell surface receptor on TAM responsible for the binding of CSSTRESAC (SEQ ID NO:1). To determine whether PDIA3 is present on the cell surface of TAM in TNBC, CD11b⁺ TAM isolated from EF43.fgf4 tumors was co-stained with antibodies against IL-10, IL-12, and PDIA3. Flow cytometry analysis showed robust expression of PDIA3 on the surface of CD11 b⁺IL-10^{high}IL-12^{low} TAM (FIG. 6A), identifying PDIA3 as a new cell membrane-associated candidate marker of M2-polarized macrophages. Consistently, EF43.fgf4 cells isolated from tumors did not express PDIA3 (FIG. 6B), in agreement with the lack of CSSTRESAC-phage binding to EF43.fgf4 cells. Moreover, immunofluorescence staining of frozen breast tumor sections from tumor-bearing mice receiving CSSTRESAC-phage iv suggested co-localization between PDIA3 and CD68, a well-established cell surface marker of macrophages (FIGS. 6C and 6D). Finally, administration of an anti-PDIA3 antibody into EF43.fgf4 tumor-bearing mice confirmed accessibility of PDIA3 through the systemic circulation (FIG. 7A). Notably, extracellular expression of PDIA3 was largely restricted to resident macrophage in tumors, while control tissues showed minimal cell surface staining. The macrophage marker F4/80 served as an additional positive control (FIG. 7B).

### CSSTRESAC (SEQ ID NO: 1) Mimics Active Vitamin D, Binds to DBP and Mediates Activation of PDIA3 on the Surface of TAMs

To gain insight into the biological mechanisms associated with this newly discovered ligand-receptor system, it was next evaluated whether the predicted interactions between CSSTRESAC (SEQ ID NO:1) and PDIA3 on the surface of TAM would have functional consequences. CD11b⁺F4/80⁺ TAM was isolated from EF43.fgf4 mammary tumors, established in culture (>99% purity by FACS), and cytokine production was tested as a surrogate for immunoregulatory responses upon treatment (FIGS. 6E-6G). Cytokines were measured by real-time quantitative PCR after RNA extraction from cultured CD11b⁺F4/80⁺ TAM exposed to soluble CSSTRESAC. Untreated cultured CD11b⁺F4/80⁺ TAM served as negative controls. Treatment of CD11b⁺F4/80⁺ TAM with the soluble CSSTRESAC (SEQ ID NO:1) peptide induced a marked (on average ˜40-fold) increase in gene expression of the pro-inflammatory cytokines IL-1β, TNF-α, and IL-6 (FIGS. 6F and 6G). In contrast, there were much lower increases in gene expression of the anti-inflammatory cytokines TGF-β1, TGF-β2, IL-10, and arginase-1 (FIGS. 6E-6G) with IL-4 and IL-13 being undetectable. iNOS₂ (˜20-fold) and the cytokine IL-23 (˜10-fold) were also substantially increased upon exposure to CSSTRESAC (SEQ ID NO:1). IL-18, IL-12, and INFβ showed modest increases or were detected only at background levels (FIGS. 6F and 6G; FIG. 7C). This cellular response was abrogated when CSSTRESA-treated CD11b⁺F4/80⁺ TAM were co-treated with 1,25-(OH)₂D₃ (FIGS. 6F and 6G), verifying that it was specifically caused by the binding of the CSSTRESAC (SEQ ID NO:1) peptide. Thus, binding of CSSTRESAC (SEQ ID NO:1) directly to TAM may alter the local anti-tumor immune response through changes in cytokine production.

### Targeted Ablation of PDIA3-Expressing TAMs Affects Tumor Growth

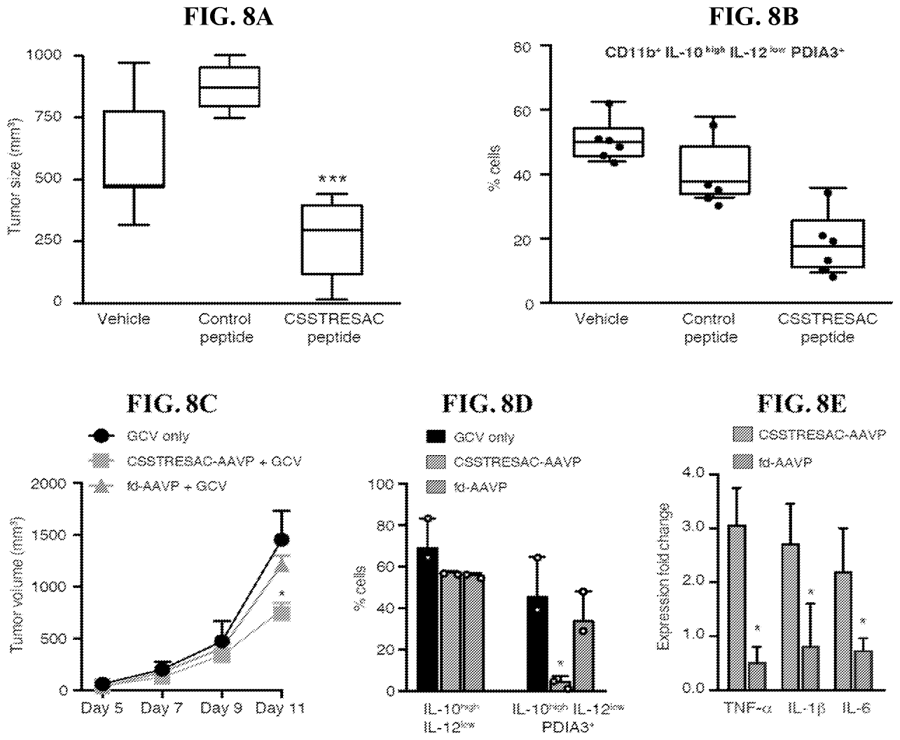

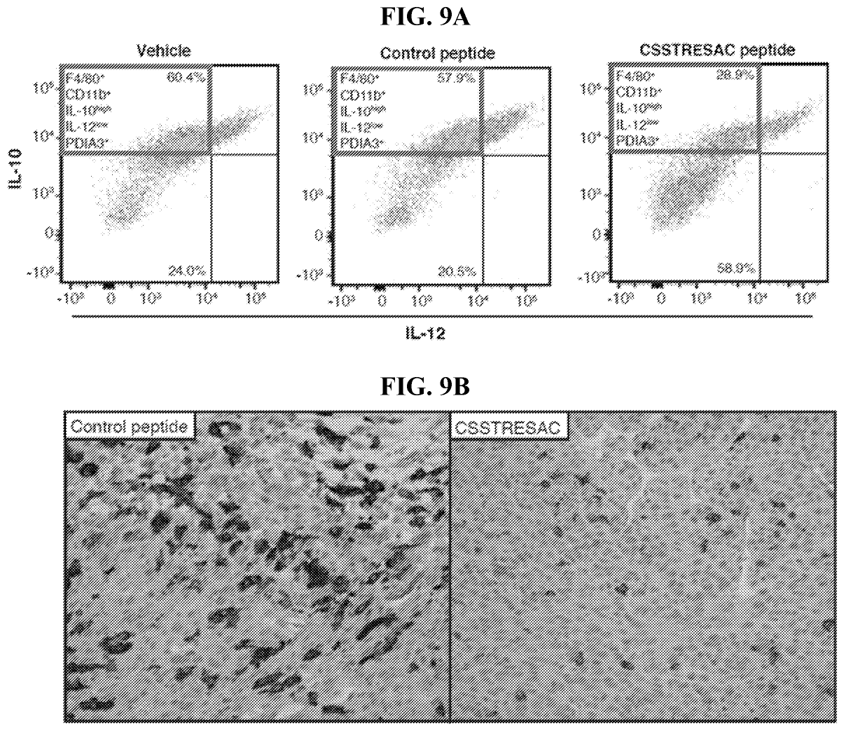

The biological significance and potential therapeutic effects of CSSTRESAC (SEQ ID NO:1) in the EF43.fgf4 tumor model was next investigated (FIG. 8A). Mice bearing size-matched EF43.fgf4 tumors were treated iv with soluble CSSTRESAC (SEQ ID NO:1) peptide, unrelated control peptide, or vehicle. A significant delay in tumor growth of mice treated with CSSTRESAC was observed as soon as one-week post initiation of treatment, compared to tumors of mice receiving an unrelated control peptide or vehicle alone (FIG. 8A, t-test, P<0.001). FACS analysis of CD11b⁺F4/80⁺ TAM showed a reduction in the number of CD11b⁺IL10^{high}IL12^{low} PDIA3-expressing TAM in tumors from mice treated with soluble CSSTRESAC (SEQ ID NO:1) peptide as compared to the negative control groups (FIG. 8B and FIG. 9A). Immunohistochemistry staining of representative tumor sections further demonstrated a reduction of the macrophage population in tumors treated with soluble CSSTRESAC (SEQ ID NO:1) (FIG. 9B). Thus, treatment of tumors with the soluble CSSTRESAC (SEQ ID NO:1) peptide inhibited tumor growth and altered the TAM population in tumors, which supports it as a potential anti-tumor drug lead candidate.

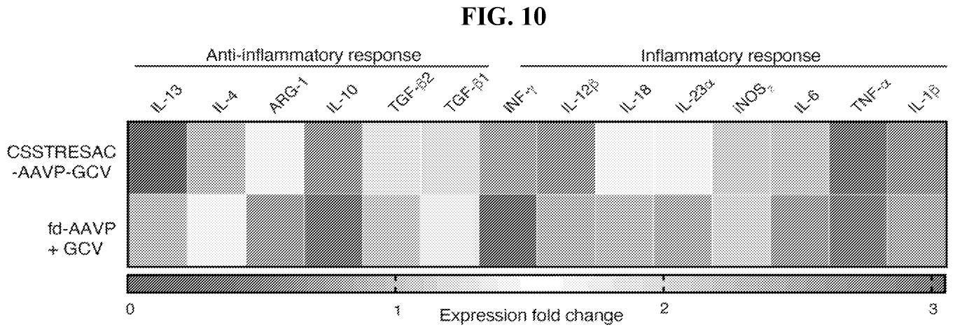

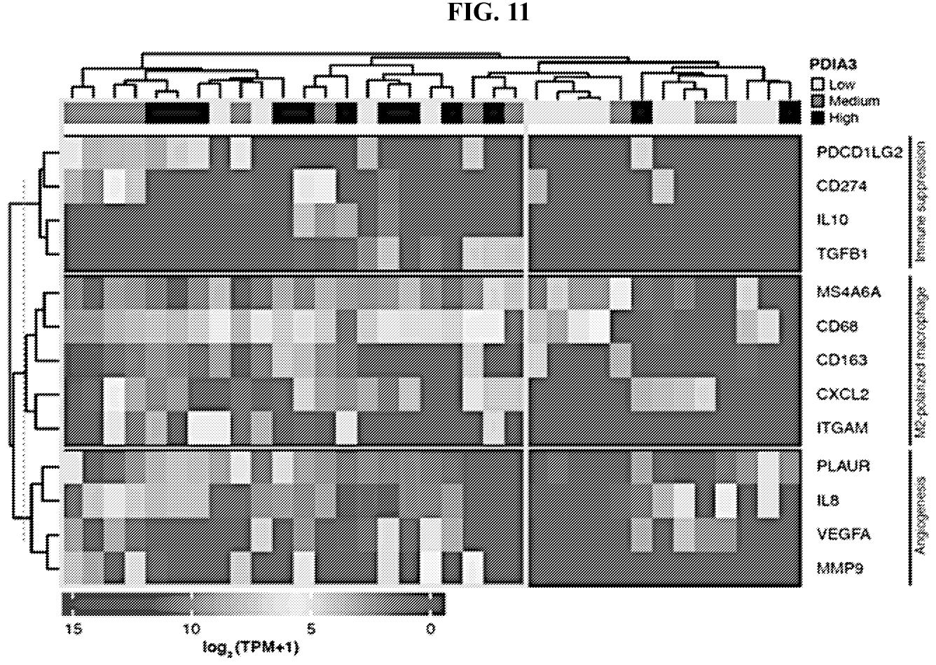

As an additional medical application, the use of CSSTRESAC (SEQ ID NO:1) as a theranostic ligand for targeting transgenes directly to tumors in preclinical settings was also analyzed. Adeno-associated/phage (AAVP) constructs were synthesized carrying the Herpes simplex virus thymidine kinase (HSVtk) gene to enable targeted suicide therapy upon combination with the pro-drug ganciclovir (GCV). CSSTRESAC-AAVP-HSVtk or control AAVP lacking the targeting peptide (fd-AAVP-HSVtk) were delivered to cohorts of size-matched EF43.fgf4 tumor-bearing mice. Animals treated with vehicle were used as controls (n=10, each cohort). All cohorts received GCV. By the end of the experiment, the sizes of tumors in mice that received CSSTRESAC-AAVP-HSVtk were significantly smaller than that of mice receiving control fd-AAVP-HSVtk or vehicle alone (FIG. 8C, t-test, P<0.001). Moreover, macrophage quantification showed a reduction in the number of F480⁺CD11b⁺IL10^{high}IL12^{low} PDIA3-expressing TAM (FIG. 8D) accompanied by a shift in the cytokine profile toward an inflammatory response in the tumor microenvironment (FIG. 8E and FIG. 10).

To evaluate whether the experimental findings in mouse models might have medical relevance for human patients, a publicly available single-cell transcriptome dataset of breast cancer and immune-infiltrating cells containing data from TNBC patients for PDIA3-expressing TAM was searched. Transcripts per million reads (TPM), single-cell (sc)RNA-seq and sample information were obtained from the Gene Expression Omnibus (GEO) repository (accession #GSE75688); an initial gene set variation (GSVA) analysis extracted single cells (n=35) displaying gene expression pathways of infiltrating macrophages. Expression of the PDIA3 gene in these cells was deemed high, medium, or low and was clustered/plotted relative to the expression of established markers of immune suppression and M2-polarized macrophages (IL-10, TGFB1, CD274, PDCDILG2, CD68, CD163, ITGAM, CXCL2, and MS4A6A). Markers of angiogenesis and/or disease progression (PLAUR, IL8, VEGFA, and MMP9) were also included. An unsupervised clustering analysis (FIG. 11) showed that high levels of PDIA3 expression in TAM clustered positively with markers of M2-polarized TAM as well as poor prognosis indicators and genes associated with immune suppression. These genomic results support the presence of PDIA3-expressing TAM in human TNBC, and indicate that these preclinical findings are clinically meaningful.

### CSSTRESAC-AAVP-TNF In Vivo in EF43-Tumor-Bearing Mice

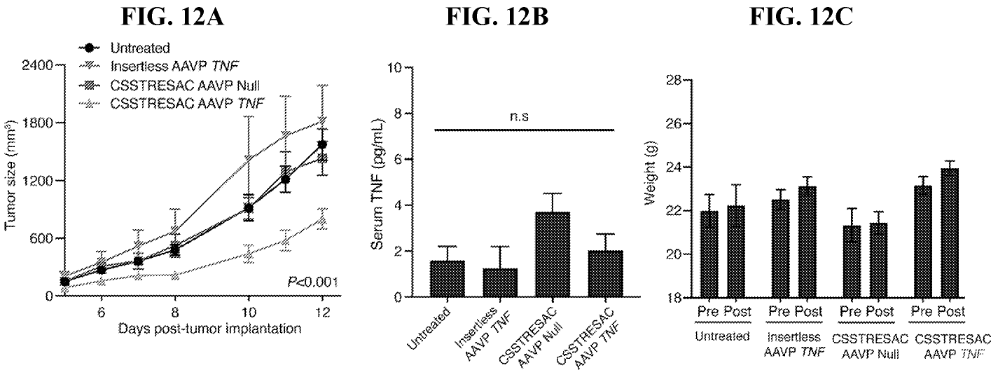

CSSTRESAC-AAVP-TNF (n=10) or the controls lacking the targeting peptide (insertless AAVP-TNF; n=5) or lacking the TNF transgene (CSSTRESAC-AAVP-Null; n=5) were divided into cohorts of size-matched EF43.fgf4 tumor-bearing mice. Untreated tumor-bearing mice were used as controls (n=5). By the end of the experiment, the sizes of tumors in mice that received CSSTRESAC-AAVP-TNF were significantly smaller than that of mice receiving insertless AAVP-TNF or CSSTRESAC-AAVP-Null controls (FIG. 12A, two ways ANOVA p<0.001). Moreover, we measured the presence of soluble TNF in the sera of animals treated with CSSTRESAC-AAVP-TNF or the controls. No difference was observed among the groups which would suggest site-specific expression of TNF (FIG. 12B). This result was supported by bodyweight measurements, which indicate no sign of cytokine-induced toxicity in any treatment groups (FIG. 12C). Without wishing to be bound by theory, these results indicate that CSSTRESAC-AAVP-TNF delayed tumor growth in this experimental setting.

### Selected Discussion

It is reported herein that PDIA3 is a functional receptor expressed on the cell surface of the M2-like class of TAM in TNBC. It is shown that PDIA3, an established vitamin D-interacting protein, has immunoregulatory functions as the TAM cell surface receptor for the peptide CSSTRESAC (SEQ ID NO:1), with clear effects in preclinical non-TNBC and TNBC mouse models and likely in TNBC patients. Therefore, CSSTRESAC (SEQ ID NO:1) can be used as a non-steroidal vitamin D analogue prototype for drug lead-optimization. Finally, a ligand-directed AAVP-HSVtk platform for theranostics is introduced based on cell surface targeting of PDIA3. The inclusion of a theranostic transgene (HSVTk) enables a personalized approach for each tumor. After AAVP administration, the patient can be imaged to identify the day of maximum TK expression. Ganciclovir treatment can be initiated then, followed by additional imaging studies to evaluate the efficacy of the GCV therapy.

These observations in vitro, in mouse mammary tumor models, plus an initial in silico analysis of cells from TNBC patients, support an unrecognized regulatory role of PDIA3-expressing TAM in the tumor immune response. Although not wishing to be limited by theory, it is believed that the CSSTRESAC-DBP complex specifically binds to PDIA3 and elicits functional changes in PDIA3-expressing TAM within the tumor microenvironment. Such biochemical and cellular alterations result in an inflammatory local response potentially mediated by IL-6, IL--1β, and TNF-α, and inhibition of tumor growth.

### ENUMERATED EMBODIMENTS

The following enumerated embodiments are provided, the numbering of which is not to be construed as designating levels of importance.

Embodiment 1 provides a tumor associated macrophage (TAM) targeting peptide comprising at least one amino acid sequence selected from the group consisting of CSSTRESAC (SEQ ID NO:1), CRYSAARSC (SEQ ID NO:2), CRGFVVGRC (SEQ ID NO:3), and CQRALMIAC (SEQ ID NO:4).

Embodiment 2 provides the TAM targeting peptide of embodiment 1, which comprises the amino acid sequence of SEQ ID NO:1.

Embodiment 3 provides the TAM targeting peptide of embodiment 1, which consists of an amino acid sequence selected from the group consisting of SEQ ID NOs:1-4.

Embodiment 4 provides a solid particle, wherein the surface of the solid particle displays the TAM targeting peptide of any one of claims 1-3, wherein the solid particle is selected from the group consisting of a bacteriophage, engineered cell, tissue fragment, nanoparticle, vesicle, dendrimer, virus-like particle, adenovirus, adeno-associated virus (AAV), adeno-associated virus phage (AAVP), and any combinations thereof.

Embodiment 5 provides the solid particle of embodiment 4, wherein the TAM targeting peptide is attached to and/or displayed on the surface of the solid particle.

Embodiment 6 provides the solid particle of embodiment 4, which further comprises an agent selected from the group consisting of a therapeutic agent, biologically active molecule, imaging agent, radioactive agent, salt, peptide, protein, lipid, nucleic acid, gas, and any combinations thereof, wherein the agent is attached to and/or contained within the solid particle.

Embodiment 7 provides the solid particle of embodiment 4, wherein the solid particle is an AAVP.

Embodiment 8 provides the solid particle of embodiment 7, wherein the AAVP comprises a therapeutic or suicide gene.

Embodiment 9 provides the solid particle of embodiment 8, wherein the therapeutic gene is tumor necrosis factor (TNF).

Embodiment 10 provides the solid particle of embodiment 8, wherein the suicide gene is Herpes simplex virus thymidine kinase (HSVtk).

Embodiment 11 provides a method of targeting a solid particle to a tumor associated macrophage (TAM) in a subject, the method comprising
administering to the subject the solid particle,
wherein the TAM targeting peptide of any one of claims 1-3 is attached to and/or displayed on the surface of the solid particle,
wherein the solid particle is selected from the group consisting of a bacteriophage, engineered cell, tissue fragment, nanoparticle, vesicle, dendrimer, virus-like particle, adenovirus, adeno-associated virus (AAV), adeno-associated virus phage (AAVP), and any combinations thereof.

Embodiment 12 provides the method of embodiment 10, wherein the TAM targeting peptide comprises the amino acid sequence of SEQ ID NO:1.

Embodiment 13 provides the method of embodiment 10, wherein the TAM targeting peptide consists of an amino acid sequence selected from the group consisting of SEQ ID NOs:1-4.

Embodiment 14 provides the method of embodiment 10, wherein the solid particle further comprises an agent selected from the group consisting of a therapeutic agent, biologically active molecule, imaging agent, radioactive agent, salt, peptide, protein, lipid, nucleic acid, gas, and any combinations thereof, wherein the agent is attached to and/or contained within the solid particle.

Embodiment 15 provides the method of embodiment 10, wherein the solid particle is an AAVP.

Embodiment 16 provides the method of embodiment 14, wherein the AAVP comprises a therapeutic or suicide gene.

Embodiment 17 provides the method of claim 16, wherein the therapeutic gene is tumor necrosis factor (TNF).

Embodiment 18 provides the method of embodiment 15, wherein the suicide gene is Herpes simplex virus thymidine kinase (HSVtk).

Embodiment 19 provides a method of treating, killing, and/or preventing growth of a tumor infiltrated with a tumor associated macrophage (TAM) in a subject,
the method comprising administering to the subject a solid particle, wherein the TAM targeting peptide of any one of claims 1-3 is attached to and/or displayed on the surface of the solid particle,
wherein the solid particle is selected from the group consisting of a bacteriophage, engineered cell, tissue fragment, nanoparticle, vesicle, dendrimer, virus-like particle, adenovirus, adeno-associated virus (AAV), adeno-associated virus phage (AAVP), and any combinations thereof.

Embodiment 20 provides the method of embodiment 17, wherein the TAM targeting peptide comprises the amino acid sequence of SEQ ID NO:1.

Embodiment 21 provides the method of embodiment 17, wherein the TAM targeting peptide consists of an amino acid sequence selected from the group consisting of SEQ ID NOs:1-4.

Embodiment 22 provides the method of embodiment 17, wherein the solid particle further comprises an agent selected from the group consisting of a therapeutic agent, biologically active molecule, imaging agent, radioactive agent, salt, peptide, protein, lipid, nucleic acid, gas, and any combinations thereof, wherein the agent is attached to and/or contained within the solid particle.

Embodiment 23 provides the method of embodiment 17, wherein the solid particle is an AAVP which comprises a therapeutic or suicide gene.

Embodiment 24 provides the method of claim 23, wherein the therapeutic gene is tumor necrosis factor (TNF).

Embodiment 25 provides the method of embodiment 21, wherein the gene comprises the Herpes simplex virus thymidine kinase (HSVtk) gene.

Embodiment 26 provides the method of embodiment 22, wherein the method further comprises:
monitoring the tumor for elevated thymidine kinase expression; and
administering a prodrug selected from ganciclovir, ganciclovir elaidic acid ester, penciclovir, acyclovir, valacyclovir, (E)-5-(2-bromovinyl)-2′-deoxyuridine, zidovuline, 2′-exo-methanocarbathymidine, and combinations thereof to the subject when elevated thymidine kinase expression is detected in the tumor.

Embodiment 27 provides the method of embodiment 23, wherein the method further comprises evaluating the efficacy of the prodrug in treating, killing, and/or preventing growth of the tumor.

### OTHER EMBODIMENTS

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

The disclosures of each and every patent, patent application, and publication cited herein are hereby incorporated herein by reference in their entirety. While this disclosure has been disclosed with reference to specific embodiments, it is apparent that other embodiments and variations of this disclosure can be devised by others skilled in the art without departing from the true spirit and scope of the disclosure. The appended claims are intended to be construed to include all such embodiments and equivalent variations.

1. A tumor associated macrophage (TAM) targeting peptide comprising at least one amino acid sequence selected from the group consisting of CSSTRESAC (SEQ ID NO:1), CRYSAARSC (SEQ ID NO:2), CRGFVVGRC (SEQ ID NO:3), and CQRALMIAC (SEQ ID NO:4).

2. (canceled)

3. The TAM targeting peptide of claim 1, which consists of an amino acid sequence selected from the group consisting of SEQ ID NOs:1-4.

4. A solid particle, wherein the surface of the solid particle displays the TAM targeting peptide of claim 1,
wherein the solid particle is selected from the group consisting of a bacteriophage, engineered cell, tissue fragment, nanoparticle, vesicle, dendrimer, virus-like particle, adenovirus, adeno-associated virus (AAV), adeno-associated virus phage (AAVP), and any combinations thereof,
optionally wherein the TAM targeting peptide is attached to or displayed on the surface of the solid particle.

5. (canceled)

6. The solid particle of claim 4,
which further comprises an agent selected from the group consisting of a therapeutic agent, biologically active molecule, imaging agent, radioactive agent, salt, peptide, protein, lipid, nucleic acid, gas, and any combinations thereof,
wherein the agent is attached to or contained within the solid particle.

7. (canceled)

8. The solid particle of claim 4, wherein the AAVP comprises a therapeutic or suicide gene.

9. The solid particle of claim 8, wherein at least one applies:
(a) the therapeutic gene encodes tumor necrosis factor (TNF);
(b) the suicide gene encodes Herpes simplex virus thymidine kinase (HSVtk).

10. (canceled)

11. A method of targeting a solid particle to a tumor associated macrophage (TAM) in a subject, the method comprising
administering to the subject the solid particle,
wherein the TAM targeting peptide of claim 1 is attached to or displayed on the surface of the solid particle,
wherein the solid particle is selected from the group consisting of a bacteriophage, engineered cell, tissue fragment, nanoparticle, vesicle, dendrimer, virus-like particle, adenovirus, adeno-associated virus (AAV), adeno-associated virus phage (AAVP), and any combinations thereof.

12. (canceled)

13. The method of claim 11, wherein the TAM targeting peptide consists of an amino acid sequence selected from the group consisting of SEQ ID NOs:1-4.

14. The method of claim 11, wherein the solid particle further comprises an agent selected from the group consisting of a therapeutic agent, biologically active molecule, imaging agent, radioactive agent, salt, peptide, protein, lipid, nucleic acid, gas, and any combinations thereof, wherein the agent is attached to and/or contained within the solid particle.

15. (canceled)

16. The method of claim 11, wherein the AAVP comprises a therapeutic or suicide gene.

17. The method of claim 16, wherein at least one of the following applies:
(a) the therapeutic gene encodes tumor necrosis factor (TNF);
(b) the suicide gene encodes Herpes simplex virus thymidine kinase (HSVtk).

18. (canceled)

19. A method of treating, killing, and/or preventing growth of a tumor infiltrated with a tumor associated macrophage (TAM) in a subject,
the method comprising administering to the subject a solid particle, wherein the TAM targeting peptide of claim 1 is attached to or displayed on the surface of the solid particle,
wherein the solid particle is selected from the group consisting of a bacteriophage, engineered cell, tissue fragment, nanoparticle, vesicle, dendrimer, virus-like particle, adenovirus, adeno-associated virus (AAV), adeno-associated virus phage (AAVP), and any combinations thereof.

20. (canceled)

21. The method of claim 19, wherein the TAM targeting peptide consists of an amino acid sequence selected from the group consisting of SEQ ID NOs:1-4.

22. The method of claim 19, wherein the solid particle further comprises an agent selected from the group consisting of a therapeutic agent, biologically active molecule, imaging agent, radioactive agent, salt, peptide, protein, lipid, nucleic acid, gas, and any combinations thereof, wherein the agent is attached to and/or contained within the solid particle.

23. The method of claim 19, wherein the solid particle is an AAVP which comprises a therapeutic or suicide gene.

24. The method of claim 23, wherein at least one of the following applies:
(a) the therapeutic gene encodes tumor necrosis factor (TNF), optionally wherein the method further comprises monitoring the tumor for elevated TNF expression;
(b) the suicide gene encodes Herpes simplex virus thymidine kinase (HSVtk).

25. (canceled)

26. The method of claim 19, wherein the method further comprises:
monitoring the tumor for elevated thymidine kinase expression; and
administering a prodrug selected from ganciclovir, ganciclovir elaidic acid ester, penciclovir, acyclovir, valacyclovir, (E)-5-(2-bromovinyl)-2′-deoxyuridine, zidovuline, 2′-exo-methanocarbathymidine, and combinations thereof to the subject when elevated thymidine kinase expression is detected in the tumor;
optionally evaluating the efficacy of the prodrug in treating, killing, or preventing growth of the tumor.

27. (canceled)

28. (canceled)

29. A method of treating, killing, or preventing growth of a tumor infiltrated with a tumor associated macrophage (TAM) in a subject, the method comprising administering to the subject an effective amount of a TAM targeting peptide.

30. The method of claim 29, wherein the TAM targeting peptide comprises the amino acid sequence of SEQ ID NO:1.

31. The method of claim 29, wherein the TAM targeting peptide comprises an amino acid sequence selected from the group consisting of SEQ ID NO: 1-4.


SEQUENCE LISTING (4 sequences)

SEQ ID NO: 1 - protein, 9 aa, Artificial Sequence
  TAM Targeting Peptide
CSSTRESAC

SEQ ID NO: 2 - protein, 9 aa, Artificial Sequence
  TAM Targeting Peptide
CRYSAARSC

SEQ ID NO: 3 - protein, 9 aa, Artificial Sequence
  TAM Targeting Peptide
CRGFVVGRC

SEQ ID NO: 4 - protein, 9 aa, Artificial Sequence
  TAM Targeting Peptide
CQRALMIAC